(19) United States
(12) Patent Application Publication
(10) Pub. No.: US 2026/0337187 A1
(43) Pub. Date: Oct. 29, 2026

(54) COMPOUNDS FOR TREATMENT OF INFLAMMATORY PROCESSES

(71) Applicant: THORNE LIMITED, Vancouver (CA)

(72) Inventors: Viktoria BETANELI, Dresden (DE); Pearce GLEN, Dresden (DE); Rolf JESSBERGER, Dresden (DE); Jirí ŠTOHANDL, Vancouver (CA); Jaroslav FRANTIŠEK, Vancouver (CA); Sergii OKOROCHENKOV, Vancouver (CA); Aleš BRUKNER, Vancouver (CA); Lukáš KUCHAR, Vancouver (CA); Jana ŠINDELÁROVÁ, Vancouver (CA); Roland R. FRANKE, Vancouver (CA); Petr SOVA, Vancouver (CA); Kang HAN, Vancouver (CA)

(73) Assignee: THORNE LIMITED, Vancouver, BC (CA)

(21) Appl. No.: 19/113,325

(22) PCT Filed: Sep. 19, 2023
(86) PCT No.: PCT/EP2023/075782

§ 371 Date: Mar. 19, 2025

Related U.S. Application Data
(60) Provisional application No. 63/408,305, filed on Sep. 20, 2022.
(60) Provisional application No. 63/408,624, filed on Sep. 21, 2022.

(51) Int. Cl. C07D 417/12; A61K 31/428

(52) U.S. Cl. CPC C07D 417/12; A61K 31/428

(57) ABSTRACT

The present disclosure relates to the use of compounds such as compounds of Formula I or a pharmaceutically acceptable salt, solvate and/or prodrug thereof, for example, in pharmaceutical compositions and in the treatment of diseases, disorders or conditions such as diseases, disorders or conditions treatable by inhibiting SWAP-70 and/or treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration, such as treatment of lung inflammation induced by viruses such as SARS-CoV-2:

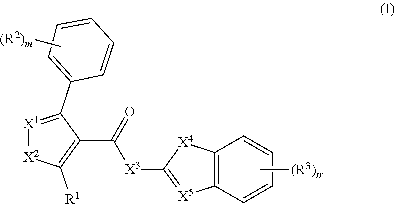

### CROSS-REFERENCE TO RELATED APPLICATIONS

The present application claims the benefit of priority from co-pending U.S. provisional application No. 63/408,305 filed on Sep. 20, 2022, and U.S. provisional application No. 63/408,624 filed on Sep. 21, 2022, the contents of both of which are incorporated herein by reference in their entirety.

### FIELD

The present disclosure relates to the use of compounds such as 3-(2-chlorophenyl)-5-methyl-N-(6-nitrobenzo[d]thiazol-2-yl)isoxazole-4-carboxamide (B2), for example, in pharmaceutical compositions and in the treatment of diseases, disorders or conditions such as those treatable by inhibiting SWAP-70 and/or treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration, such as treatment of lung inflammation induced by viruses such as SARS-CoV-2.

### BACKGROUND

SWAP-70 was first described in 1998 as a protein involved in the isotype switch of immunoglobulins (Borggrefe, Wabl et al. 1998), and in 2000 as a protein which in murine B cells and in mice supports the production specifically of the IgE isotype of immunoglobulins (Borggrefe, Keshavarzi et al. 2001). In subsequent studies, it was shown that SWAP-70 also supports mast cell degranulation, which is the key effector process in allergic reactions (Gross, Borggrefe et al. 2002, Sivalenka and Jessberger 2004, Sivalenka, Sinha et al. 2008). Further, a key role of the protein in hematopoietic cell migration and adhesion, which are central to many inflammatory processes (Pearce, Angeli et al. 2006, Chopin, Quemeneur et al. 2010, Chopin, Chacon-Martinez et al. 2011, Ocana-Morgner, Reichardt et al. 2011, Pearce, Audzevich et al. 2011) was demonstrated. The underlying mechanisms were then identified: in the cytoplasm and at cytoplasmic membranes SWAP-70 controls specific F-actin rearrangements in certain hematopoietic cells and thereby controls cell morphogenesis, mobility, adhesion and several specific functions that depend on the F-actin network (Chacon-Martinez, Kiessling et al. 2013, Betaneli and Jessberger 2020). In addition, in B cells SWAP-70 enters the nucleus and regulates the antagonistic action of transcription factors which modulate IgE production (Audzevich, Pearce et al. 2013). The cell types whose disease-relevant activities are controlled by SWAP-70 include B lymphocytes of several stages of development including plasma cells and plasmablasts, mast cells, neutrophils, eosinophils, macrophages, osteoclasts, and endothelial cells.

US Patent Application Publication No. 2003/0166018 discloses screening methods to identify agents that modulate a level or an activity of a SWAP-70 protein in a degranulation competent cell. Canadian Patent No. 2,796,171 discloses a method for identifying a substance that inhibits IgE production, the method comprising: contacting at least one candidate substance, SWAP-70, and BCL-6 and/or STAT-6 and selecting a candidate substance which specifically inhibits SWAP-70 mediated activation of STAT-6 and/or reinforces the BCL-6 mediated inhibition of SWAP-70 mediated activation of STAT-6. Canadian Patent Application No. 2,900,798 discloses a method for identifying a substance which inhibits the activity of SWAP-70, the method comprising: contacting at least one test substance with SWAP-70, detecting the degree of dimerization of SWAP-70, and selecting a test substance which inhibits the dimerization of SWAP-70.

The allergic reaction, a widespread and increasing disease of mild to life-threatening forms, is caused by much increased production of the IgE isotype of antibodies, which binds to an allergen (e.g., hay pollen, nut compounds, insect venom components and many others). IgE is bound to the surface of mast cells through a high-affinity receptor and upon allergen binding triggers an immediate and very strong reaction of mast cells: the sudden release of greater than 200 preformed biologically active molecules (histamine, cytokines, leukotrienes, etc.), which cause the allergic reaction. While there are treatments that soften the symptoms (e.g., histamine antagonists and corticosteroids), there is no treatment that prevents the underlying cause, the production of IgE. It has been shown that SWAP-70 promotes IgE production and the respective mechanism clarified (Borggrefe, Keshavarzi et al. 2001, Audzevich, Pearce et al. 2013) in murine splenic B cells and in vivo in mice. In addition, SWAP-70 supports mast cell degranulation; in the absence of SWAP-70, murine mast cells are much impaired in IgE-triggered degranulation, and SWAP-70 deficient mice show much reduced local and systemic IgE-triggered anaphylaxis (Gross, Borggrefe et al. 2002, Sivalenka, Sinha et al. 2008).

Lower respiratory tract infections by viruses and/or bacteria are a leading cause of infection-related death. In disease caused by the SARS-CoV-2 virus (COVID-19), but also in other viral infections such as respiratory syncytial virus (RSV) and influenza infections, an acute and life-threatening inflammation of the lungs is triggered, typically a few days after onset of symptoms (Zaki and Paddock 2008, Merad, Blish et al. 2022). In the case of COVID-19, this inflammation features massively increased pro-inflammatory cytokine levels (“cytokine storm”), depletion of T cells and some dendritic cell types, excessive expansion of plasmablasts, appearance of auto-antibodies that target type I interferons reducing their anti-viral effect, and hyper-activation of immune cells such as neutrophils, which damage lung airways through excessive NETosis and/or reactive oxygen species (ROS) production. Additional pathologic responses include mast cell activation, IgE production, endothelial inflammation and blood clotting, and hyper-activated macrophage responses causing lung fibrosis (Bastard, Rosen et al. 2020, Fajgenbaum and June 2020, Kuri-Cervantes, Pampena et al. 2020, Lucas, Wong et al. 2020, Mathew, Giles et al. 2020, Merad and Martin 2020, Veras, Pontelli et al. 2020, Ferreira-Gomes, Kruglov et al. 2021, Gustine and Jones 2021, Wendisch, Dietrich et al. 2021, Wu, Liu et al. 2021, Merad, Blish et al. 2022). Several drugs were developed to directly suppress COVID-19 virus proliferation but need to be administered early in the course of the infection to be effective. General immunosuppressants are administered with limited success [https://www.covid19treatmentguidelines.nih.gov]. These drugs, e.g., corticosteroids like dexamethasone, carry various risks such as triggering diabetes and lowering the defense against pathogens. Corticosteroids need to be taken for prolonged times (4-8 weeks), reach their maximal effect only after about 2 weeks, and the dose has to be slowly reduced, typically for 1-3 weeks. Inhibitors of the IL6 receptor and of the JAK-STAT pathway are also used in therapy of patients that show indications of active cytokine storm and excessive inflammation with improvement in a relatively small percentage of such patients. To our knowledge, no effective and safe immune modulator that prevents hyper-inflammation has been reported and applied for COVID-19 therapy and in related viral or non-viral inflammatory diseases. Clearly, a new drug would be desirable to address the severe and variable inflammatory cascades happening in patients with COVID-19. The same is true for other virus-induced inflammation of the lung, where innovative and novel drugs are desirable (Zaki and Paddock 2008). Non-infectious pulmonary conditions such as asthma or chronic obstructive pulmonary disease (COPD) can be exacerbated by certain viral infections such as infections with RSV, influenza, rhinovirus, parainfluenza, and/or coronaviruses (Kennedy, Pham et al. 2019). These infections can, for example, cause acute respiratory distress syndrome (ARDS), hospitalization, and sometimes death linked to inflammatory processes. Acute episodes of COPD are similarly characterized by excessive cytokine production. Besides lung inflammation, other cells and tissues within the organism are negatively impacted by virus-triggered inflammation. The effects—often enhanced through the cytokine storm—can reach systemic dimensions, including many cell types and tissues such as the gastrointestinal system, the kidneys, the heart, the nervous system, the skin, liver and more. In addition to viral infections, infections with other pathogens and other conditions can also cause severe lung and/or systemic pathologies where many of the cells and processes described above are involved. Examples are bacterial sepsis, infection with Yersinia pestis (plague), which triggers alveolar macrophages to produce excessive cytokines, or cytokine storm associated with autoimmune disorders (Fajgenbaum and June 2020).

In non-virus induced lung inflammatory diseases, the immune system overreacts causing life-threatening conditions. Similar to allergic reactions, many manifestations of asthma are also triggered by an IgE-allergen reaction, causing mast cell degranulation and subsequent inflammatory events, including eosinophil activation and migration. In addition, an important contribution of SWAP-70 to eosinophil migration into lungs (Bahaie, Hosseinkhani et al. 2012) has been published. Reduced eosinophil influx into asthma-induced mice and reduced adhesion of eosinophils were observed in absence of SWAP-70. In acute asthma, IgE-producing B lymphocytes, the Th2 response triggering Th lymphocytes, degranulating mast cells and activated eosinophils play major roles in promoting the disease (Martinez and Vercelli 2013). Current treatments include, for example, inhaled corticosteroids as a standard treatment used mostly for mild and moderate asthma with well-described side effects, or anti IgE (Omalizumab), anti IL-5 or anti IL-13 antibodies in severe asthma, with the clear need for additional and/or alternative therapy options.

Urticaria, a frequent skin disease, is typically caused by excessive mast cell degranulation (Church, Kolkhir et al. 2018). Histamine is released from mast cells in response to physical (heat, cold, pressure) or chemical stress of skin, to allergens (called a pseudo-allergic reaction), to excessive sunlight, to infections and/or by intolerance for example to common drugs like aspirin. Many of these stimuli are difficult to avoid. All these stimuli can trigger mast cell degranulation. A central role of SWAP-70 in mast cell degranulation in mice has been reported as described above. Recently, a role of eosinophils in promoting urticaria was also reported (Altrichter, Frischbutter et al. 2020). While mild forms of Urticaria can be treated with histamine blockers or leukotriene antagonists, patients suffer greatly from more severe forms, which can extend into the upper airways and restrict breathing (swelling of mucous membranes). In pseudo-allergic urticaria, IgE is involved and reducing IgE levels using anti IgE antibody therapy shows some success in some patients (Maurer, Altrichter et al. 2018, Wedi and Traidl 2021). Corticosteroids are often the last resort for treatment in severe cases. Thus, the need for additional therapies. Urticaria can progress from a spontaneous, acute state into a chronic state within about 6 weeks (Saini and Kaplan 2018).

Osteoporosis, which affects about 15% of the population, is caused by an imbalance between bone production and resorption. Osteoclasts, a hematopoietic cell type, resorb bone and in osteoporosis take over. This happens most often in post-menopausal women since levels of the inhibitory estrogen drop. Bone mass decreases and the bones become brittle and often break. Osteoclasts resorb bone by firmly adhering to bone surfaces, forming a resorption zone beneath their cell body. Then they secrete acid and proteases to destroy bone. To firmly adhere to bone and generate a low pH, protease-rich environment, osteoclasts need to form a “sealing ring”. This ring is based on specific F-actin structures. Without SWAP-70 osteoclasts cannot form a proper F-actin ring and therefore fail in bone resorption in cell culture and in mice (Garbe, Roscher et al. 2012, Roscher, Hasegawa et al. 2016). SWAP-70 deficient mice therefore become osteopetrotic, i.e., they show substantially increased bone mass. Increased bone mass is highly beneficial in osteoporosis. Current treatments include bisphosphonates, inhibition of cytokines/receptors (RANK system), estrogen receptor agonists, integrin antagonists, Src inhibitors, cathepsin K inhibitors, calcitonin—all with substantial side effects and/or limited therapeutic effects.

Chronic obstructive pulmonary disease (COPD) can currently not be healed but progresses with acute episodes that pose a great burden onto patients. COPD typically results in death. COPD like asthma predisposes to pneumonia. COPD involves hyper-activation of several immune cell types as well as enhanced cytokine release and thus can also be classified as a special cytokine release syndrome (Brightling and Greening 2019). Air pollution, smoking, allergens, and pathogens like bacteria or viruses can trigger COPD. Various forms of COPD exist, generally divided into neutrophil-dominated and eosinophil-dominated types. Other key immune cell types promoting COPD besides these two cell types are macrophages, mast cells and plasmablasts.

In a recent large genome-wide association study, SWAP-70 was identified as a high-risk genetic locus for rheumatoid arthritis (Kwon, Lim et al. 2020). Generally, in inflammatory arthritis pro-inflammatory macrophages, neutrophils, and several types of lymphocytes are overly activated, more are recruited into the bone joints (synovium), become activated, and a vicious pro-inflammatory cycle sets in that cannot be controlled anymore by the immune system (Jang, Kwon et al. 2022). Within the inflammatory arthritis milieu, osteoclasts increasingly form and become activated to resorb bone, adding to the deterioration of the joints. Rheumatoid arthritis (RA) is additionally characterized by production of autoantibodies by plasmablasts, which develop in this heavily pro-inflammatory environment, to which the plasmablasts further contribute. Inflammatory arthritis in a broader sense also includes gout. In gout urate crystals activate neutrophils to produce ROS, to perform NETosis and to secrete cytokines. RA is an autoimmune disease, and there are other autoimmune diseases one example being systemic lupus erythematosus, for which novel therapies are needed (Touma and Gladman 2017, Liossis and Staveri 2021). In this debilitating autoimmune disease, auto-reactive B cells including plasmablasts and neutrophil NETosis contribute substantially to pathogenesis (Frangou, Vassilopoulos et al. 2019). In addition, IgE levels are increased with auto-reactive IgE present. Recently systemic lupus, bullous pemphigoid, atopic dermatitis and other auto-immune diseases were described as auto-IgE immune diseases or even auto-allergies (Maurer, Altrichter et al. 2018).

Besides the cytokine storm known for virus-induced lung inflammation (e.g., triggered by various strains of coronavirus, influenza, and/or RSV), other cytokine release syndromes have been described (Fajgenbaum and June 2020). In this leading review it reads that “[c]ytokine storm and cytokine release syndrome are life-threatening systemic inflammatory syndromes involving elevated levels of circulating cytokines and immune-cell hyperactivation that can be triggered by various therapies, pathogens, cancers, autoimmune conditions, and monogenic disorders” (Fajgenbaum and June 2020). Immune therapies including T cell therapies, viral and bacterial infections, and sepsis, can cause cytokine storm, which is typically associated with high fever, headache, rash, diarrhea, respiratory problems and many other symptoms and can be fatal through multiple organ failure. The large spectrum of symptoms makes treatment of symptoms difficult. Rather, the underlying cause desirably should be addressed. Blocking individual pro-inflammatory cytokines such as IL-6 or its receptors with antibodies (e.g., tocilizumab) has shown limited beneficial effects in some cases and will need to be combined with standard or new therapies; blocking IL-6 can also promote infections due to long half-life of such treatments, limiting its use (Kang and Kishimoto 2021).

Inflammation associated with endothelial cell activation and cytokine release can trigger a number of diseases such as artherosclerotic plaques and thus coronary artery disease through monocyte accumulation and adhesion, vascular dysfunction, tissue infiltration of migration-activated leukocytes, and/or excessive activation of the coagulation cascade. Overshooting secretion of pro-inflammatory cytokines by endothelial cells, particularly of chemokines which stimulate pathological recruitment and accumulation of leukocytes, is a major concern. In sepsis, endothelial cytokines contribute to the massive recruitment and transendothelial migration of neutrophils, a leading cause of patient death (Kilpatrick and Kiani 2020). Similarly, neutrophils are recruited and aggregate during thrombo-inflammation. In a very large genome-wide analysis study, SWAP-70 was identified as a high risk, causal genetic locus for coronary artery disease (Nikpay, Goel et al. 2015), where the protein is suggested to promote pathological leukocyte migration and adhesion at the vessel walls. SWAP-70 was confirmed as a causal gene for coronary artery disease in a more recent meta-study (Zheng, Ma et al. 2020).

### SUMMARY

An objective of the present invention is to decrease inflammatory processes. These include inflammation-promoting activities of relevant immune cells such as mast cells, B lymphocytes, neutrophils and others, as well as pro-inflammatory reactions of endothelial cells. B2 was found to inhibit activities of certain immune cells. As B2 will not suppress the activity of the immune system as a whole, it will target immune cell activities that contribute to disease without eliminating an anti-pathogen immune response. Thus, B2 and other compounds of the present disclosure are predicted to act as immunomodulators, not global immunosuppressants.

Accordingly, the present disclosure includes a method of treating an inflammatory disease, disorder or condition, the method comprising administering an effective amount of a compound of Formula I or a pharmaceutically acceptable salt, solvate and/or prodrug thereof to a subject in need thereof:

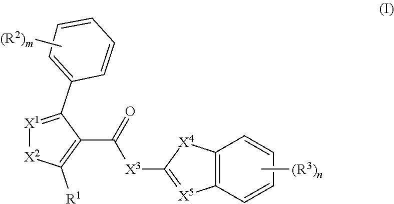

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a method of treating a disease, disorder or condition treatable by inhibiting SWAP-70, the method comprising administering an effective amount of a compound of Formula I or a pharmaceutically acceptable salt, solvate and/or prodrug thereof to a subject in need thereof:

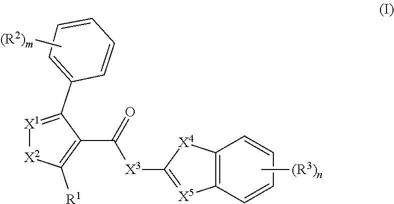

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a method of treating a disease, disorder or condition treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration, the method comprising administering an effective amount of a compound of Formula I or a pharmaceutically acceptable salt, solvate and/or prodrug thereof to a subject in need thereof:

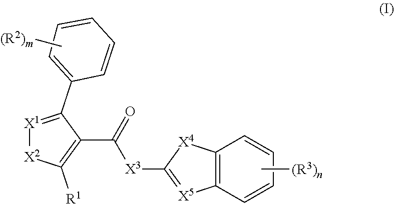

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 1 to 5; and
   n is an integer of from 1 to 4.

In an embodiment, m is 1.

In an embodiment, n is 1.

In an embodiment:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

In an embodiment, the compound of Formula I is a compound of Formula I(a):

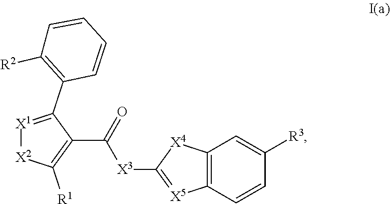

wherein R¹, R², R³, X¹, X², X³, X⁴ and X⁵ are as described herein.

In an embodiment, R³ is NO₂.

In an embodiment, R¹ is C₁₋₄alkyl. In another embodiment, R¹ is methyl.

In an embodiment, R² is halo. In another embodiment, R² is chloro.

In an embodiment, X¹ is N.

In an embodiment, X² is O.

In an embodiment, X³ is NH.

In an embodiment, X⁴ is S.

In an embodiment, X⁵ is N.

In an embodiment, the compound of Formula I has the structure:

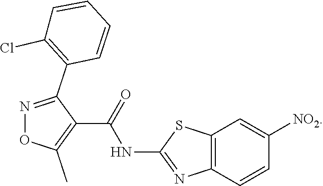 [SMILES: Cc1onc(-c2ccccc2Cl)c1C(=O)Nc1nc2ccc([N+](=O)[O-])cc2s1]

In an embodiment, the compound of Formula I or the pharmaceutically acceptable salt, solvate and/or prodrug thereof is the compound of Formula I.

In an embodiment, the subject is a human.

In an embodiment, the disease, disorder or condition is a microbial infection, asthma, systemic inflammation, endothelial inflammation, an allergy, urticaria, osteoporosis, chronic obstructive pulmonary disease (COPD), arthritis, an autoimmune disorder, a cytokine release syndrome, avascular necrosis and/or sepsis. In another embodiment, the microbial infection is a viral infection, and the treatment is for virus-induced lung inflammation. In a further embodiment, the compound of Formula I or the pharmaceutically acceptable salt thereof is for treatment of disease caused by the SARS-CoV-2 virus.

In an embodiment, the compound of Formula I is administered via inhalation.

The present disclosure also includes a compound of Formula I(b) or a pharmaceutically acceptable salt, solvate and/or prodrug thereof for use as a medicament:

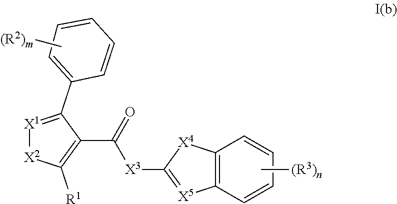

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4;
provided that:
   when m is 0, R³ is selected from substituted or unsubstituted aryl, heteroaryl, halo, NO₂ and SO₂R⁵;
   when m is 1 and R² is halo, n is an integer of from 1 to 4, and R³ is selected from substituted or unsubstituted aryl, heteroaryl and NO₂; and
   when m is 2, R² is selected from C₁₋₁₀alkyl, substituted or unsubstituted aryl, heteroaryl, OR⁴, NO₂ and SO₂R⁵.

In an embodiment, m is 1.

In an embodiment, n is 1.

In an embodiment:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

In an embodiment, the compound of Formula I(b) is a compound of Formula I(c):

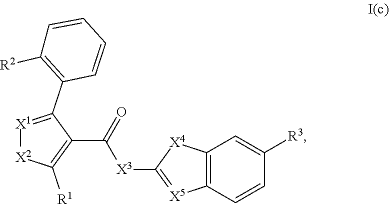

wherein R¹, R², R³, X¹, X², X³, X⁴ and X⁵ are as described herein.

In an embodiment, R³ is NO₂.

In an embodiment, R¹ is C₁₋₄alkyl. In another embodiment, R¹ is methyl.

In an embodiment, R² is halo. In another embodiment, R² is chloro.

In an embodiment, X¹ is N.

In an embodiment, X² is O.

In an embodiment, X³ is NH.

In an embodiment, X⁴ is S.

In an embodiment, X⁵ is N.

In an embodiment: R¹ is C₁₋₅alkyl, R² is halo, R³ is NO₂, X¹ is N, X² is O, X³ is NH, X⁴ is S, X⁵ is N, m is 1 and n is 1.

In an embodiment, the compound of Formula I(b) has the structure:

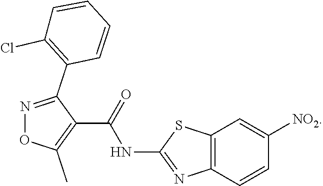 [SMILES: Cc1onc(-c2ccccc2Cl)c1C(=O)Nc1nc2ccc([N+](=O)[O-])cc2s1]

In an embodiment, the compound of Formula I(b) or the pharmaceutically acceptable salt, solvate and/or prodrug thereof is the compound of Formula I(b).

The present disclosure also includes a pharmaceutical composition comprising a compound as described herein or the pharmaceutically acceptable salt, solvate and/or prodrug thereof.

In an embodiment, the pharmaceutical composition further comprises a pharmaceutically acceptable carrier.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

The embodiments of the disclosure will now be described in greater detail with reference to the attached drawings, in which:

FIG. 1 shows inhibition of SWAP-70 dimerization in living cells by 3-(2-chlorophenyl)-5-methyl-N-(6-nitrobenzo[d]thiazol-2-yl)isoxazole-4-carboxamide (“B2”) in comparison to control: the left plot shows inhibition of the fluorescence-activated cell sorting-fluorescence resonance energy transfer (FACS-FRET) signal (FRET efficiency) and thus of SWAP-70 dimerization by three different concentrations of B2, measured at 20 minutes after stimulation of the cells; and the right plot shows inhibition by 50 μM B2 at earlier time intervals after stimulation. B2 was added to the cells 15 minutes prior to stimulation.

FIG. 2 shows the inhibition of neutrophil reactive oxygen species (ROS) production by B2 in comparison to control (far left). The effect of 4 different concentrations (from left to right: 100, 50, 20 and 10 μM) of B2 is shown. Each symbol represents one experiment.

FIG. 3 shows the inhibition of neutrophil NETosis by B2. The effect of 3 different concentrations (from left to right for each treatment: 100, 50 and 20 μM) of B2 is shown as % reduction compared to untreated control; average of 3 experiments.

FIG. 4 shows plots of % CD38+/CD19low (upper) and % CD138+(lower) as a function of concentration of B2 showing the inhibition of B lymphocyte plasmablast development by B2. The effect of two concentrations (from right to left: 20 and 10 μM) of B2 is shown in each plot in comparison to control (far left). Each symbol represents one experiment.

FIG. 5 shows inhibition of B lymphocyte IgE production by B2. B2 was applied at 3 different concentrations, from left to right: 50, 20 and 10 μM. Average of >10 experiments is shown.

FIG. 6 shows inhibition of mast cell degranulation by B2. Three different concentrations of B2 were tested (from left to right: 100, 50 and 20 μM) and the average of 11 experiments is shown as % reduction in degranulation (left plot). Degranulation was also measured at 3 time points (from left to right: 30, 15 and 5 minutes) after addition of 100 μM B2 (right plot).

FIG. 7 shows inhibition of eosinophil ROS production by B2. The effect of 3 different concentrations (from right to left: 100, 50 and 25 μM) of B2 is shown in comparison to control (far left) and ROS triggered control (PMA; second from left); average of 3 experiments.

FIG. 8 is a plot showing average relative intensity of cytokine levels relative to control showing the modulation of mast cell cytokine release by B2. Average of 3 experiments.

FIG. 9 shows the modulation of B lymphocyte cytokine release by B2. The % reduction compared to the stimulated control is shown; average of 2 experiments.

FIG. 10 shows the inhibition of macrophage/monocyte migration by B2. Dark grey: dimethylsulfoxide (DMSO) only; light grey: 50 μM B2. An exemplary experiment out of four is shown.

FIG. 11 shows the inhibition of neutrophil adhesion to endothelial cells. The number of adhered green-fluorescent labeled neutrophils per field of view normalized to the area covered by endothelial cells per field of view is shown. Three concentrations of B2 are shown next to the solvent (DMSO) control. An exemplary experiment out of three is shown.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps. As used herein, the word “consisting” and its derivatives, are intended to be close ended terms that specify the presence of stated features, elements, components, groups, integers, and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

As used herein, terms of degree such as “substantially”, “about” and “approximately” mean a reasonable amount of deviation of the modified term such that the end result is not significantly changed. These terms of degree should be construed as including a deviation of at least ±5% or at least ±10% of the modified term if this deviation would not negate the meaning of the term it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is present or used.

The term “suitable” as used herein means that the selection of the particular compound and/or conditions would depend on the specific synthetic manipulation to be performed, and/or the identity of the compound(s) to be transformed, but the selection would be well within the skill of a person skilled in the art. All synthetic method steps described herein are to be conducted under conditions sufficient to provide the product shown. A person skilled in the art would understand that all reaction conditions, including, for example, reaction solvent or lack thereof, reaction time, reaction temperature, reaction pressure, reactant ratio and whether or not the reaction should be performed under an anhydrous or inert atmosphere, can be varied to optimize the yield of the desired product and it is within their skill to do so.

The expression “proceed to a sufficient extent” as used herein with reference to the reactions or method steps disclosed herein means that the reactions or method steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

The term “subject” as used herein includes all members of the animal kingdom including mammals, and optionally refers to humans. In an embodiment, the subject is human.

The term “halo” as used herein refers to a halogen atom and includes F, Cl, Br and I. In an embodiment of the present disclosure, halo is chloro.

The term “alkyl” as used herein, whether it is used alone or as part of another group, means straight or branched chain, saturated alkyl groups. The number of carbon atoms that are possible in the referenced alkyl group are indicated by the numerical prefix “Cₙ₁₋ₙ₂”. For example, the term C₁₋₁₀alkyl means an alkyl group having 1, 2, 3, 4, 5, 6, 7, 8, 9 or 10 carbon atoms.

The term “aryl” as used herein, whether used alone or as part of another group, refers to groups that contain at least one aromatic ring. When an aryl group contains more than one aromatic ring the term “aryl” as used herein includes condensed aromatic systems. In an embodiment, the aryl group contains from 6, 9, 10 or 14 atoms, such as phenyl, naphthyl, indanyl or anthracenyl. The number of carbon atoms that are possible in the referenced aryl group are indicated by the numerical prefix “Cₙ₁₋ₙ₂”. For example, the term C₆₋₁₀aryl means an aryl group having 6, 7, 8, 9 or 10 carbon atoms.

The term “heteroaryl” as used herein, whether used alone or as part of another group, refers to an aromatic, ring-containing group having one or more multivalent heteroatoms (for example, heteroatoms independently selected from N, O and S), as a part of the ring structure. In an embodiment of the present disclosure, the heteroaryl includes at least 5 and up to 20 atoms in the ring(s). Heteroaryl groups may contain more than one ring.

The term “substituted” as used herein refers to a structure, molecule or group in which one or more available hydrogen atoms are replaced with one or more other chemical groups. A person skilled in the art would readily appreciate that the number of possible substituents would depend, for example, on the particular structure, molecule or group and/or the number of available hydrogen atoms therein. For example, 1 to 5, 1 to 4, 1 to 3, 1 to 2, 5, 4, 3, 2 and/or 1 available hydrogen atoms may be replaced with the one or more other chemical groups. The term “available hydrogen atoms” as used herein refers to hydrogens that would be known to a person skilled in the art to be capable of replacement by a suitable substituent. In an embodiment, the substituents are independently selected from one or more of C₁₋₄alkyl, aryl, heteroaryl, halo, OR⁹, NO₂ and SO₂R⁹, wherein R⁹ is selected from H, C₁₋₄alkyl, aryl and heteroaryl.

The term “B2” as used herein refers to the compound 3-(2-chlorophenyl)-5-methyl-N-(6-nitrobenzo[d]thiazol-2-yl)isoxazole-4-carboxamide, having the chemical structure:

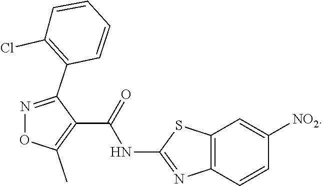 [SMILES: Cc1onc(-c2ccccc2Cl)c1C(=O)Nc1nc2ccc([N+](=O)[O-])cc2s1]

The terms “compounds of the disclosure”, “compounds of the present disclosure” and the like as used herein include compounds of Formula I and pharmaceutically acceptable salts, solvates and/or prodrugs thereof, including compounds of Formula I(a) and pharmaceutically acceptable salts, solvates and/or prodrugs thereof, including compounds of Formula I(b) and pharmaceutically acceptable salts, solvates and/or prodrugs thereof and including compounds of Formula I(c) and pharmaceutically acceptable salts, solvates and/or prodrugs thereof as well as B2 and pharmaceutically acceptable salts, solvates and/or prodrugs thereof.

The term “pharmaceutically acceptable” means compatible with the treatment of subjects, for example, mammals such as humans.

The term “pharmaceutically acceptable salt” as used herein means an acid addition salt or a base addition salt that is compatible with the treatment of subjects.

An “acid addition salt that is compatible with the treatment of subjects” is any non-toxic inorganic or organic salt of any basic compound. Basic compounds that form an acid addition salt include, for example, compounds comprising an amine group susceptible to protonation. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that may form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. The selection of a suitable salt can be made by a person skilled in the art. The formation of a desired acid addition salt is, for example, achieved using standard techniques. For example, in an embodiment of the present disclosure, the neutral compound is treated with the desired acid in a suitable solvent and the salt which is thereby formed then isolated by filtration, extraction and/or any other suitable method.

A “base addition salt that is compatible with the treatment of subjects” is any non-toxic inorganic or organic salt of any acidic compound. Acidic compounds that form a base addition salt include, for example, compounds comprising a sulfonic acid group. Inorganic bases that may form suitable salts include, without limitation, lithium, sodium, potassium, calcium, magnesium or barium hydroxide. Organic bases that may form suitable salts include, without limitation, aliphatic, alicyclic or aromatic organic amines such as methylamine, trimethylamine and picoline or ammonia. The selection of a suitable salt can be made by a person skilled in the art. The formation of a desired base addition salt is, for example, achieved using standard techniques. For example, in an embodiment of the present disclosure, the neutral compound is treated with the desired base in a suitable solvent and the salt which is thereby formed then isolated by filtration, extraction and/or any other suitable method.

The term “solvate” as used herein in reference to a compound of the disclosure refers to a complex formed between the compound and a solvent from which the compound is precipitated or in which the compound is made. Accordingly, the term “solvate” as used herein means a compound of the disclosure, wherein molecules of a suitable solvent are incorporated in the crystal lattice. Examples of suitable solvents are ethanol, water and the like. When water is the solvent, the complex is optionally referred to as a “hydrate”. The formation of solvates will vary depending on the compound and the solvate. In general, solvates are formed by dissolving the compound in an appropriate solvent and isolating the solvate by cooling or using an antisolvent and/or reducing and/or evaporating the solvent. The solvate is typically dried or azeotroped under ambient conditions. The selection of suitable conditions to form a particular solvate can be made by a person skilled in the art.

The term “prodrug” as used herein in reference to a compound of the disclosure refers to a derivative of the compound that reacts under biological conditions to provide the compound.

The term “inflammatory disease, disorder or condition” as used herein refers to a disease, disorder or condition comprising one or more inflammatory processes which are inhibited by B2 and/or other compounds of the present disclosure such as inflammatory processes in which SWAP-70 is expressed in the target cells and that are connected to activities and properties of SWAP-70. Such inflammatory processes may include, for example: (a) macrophages/monocytes: migration, phagocytosis linked to hyper-activation, excessive pro-inflammatory cytokine production, adhesion to endothelial cells (important in atherosclerosis-foam cell formation), and/or M1 pro-inflammatory macrophage development; (b) basophils: IgE-triggered degranulation contributing to allergies, asthma and other inflammatory processes, and/or excessive pro-inflammatory cytokine production; (c) endothelial cells: cytokine release, excessive ROS production, excessive proliferation and migration, aberrant activation of blood coagulation during inflammation and/or infection, chronic endothelial inflammation found in conditions with endothelial dysfunction like atherosclerosis and COPD, and/or endothelial cell-immune cell interactions, for example, immune cell adhesion to endothelial cells (in addition to neutrophils), such as monocytes in atherosclerosis, will also inhibit cell migration into tissues such as during inflammatory arthritis; (d) mast cells: degranulation, cytokine release, signaling through the FceRI, the IgE receptor, signaling through c-kit, the most important differentiation and activation receptor, and/or excessive production of pro-inflammatory, non-cytokine mediators (e.g., prostaglandins and/or leukotrienes); (e) B lymphocytes: plasmablast development, IgE production, cytokine release, Toll-like receptor triggered excessive and potentially autoreactive plasmablast formation, B-T cell synapsis formation within excessive inflammatory processes and/or B cell adhesion and migration including diapedesis through endothelium; (f) dendritic cells: excessive activation with contribution to auto-immune reactions; (g) eosinophils: ROS production, migration of eosinophils (a key process in asthma and other inflammatory conditions), degranulation and release of aggressive proteins (e.g., hydrolases, peroxidase, RNase, DNase, lipase and/or elastase), excessive production of pro-inflammatory cytokines and lipid mediators and/or adhesion of eosinophils to endothelial cells; (h) neutrophils: ROS production, NETosis, degranulation of primary, secondary and tertiary granules, excessive production of pro-inflammatory cytokines and/or migration into tissues; (i) osteoclasts: fusion of pre-osteoclasts to multi-nucleated functional osteoclasts, an important differentiation step; (j) chondroclasts: cartilage degradation by these cells, which are similar to osteoclasts, in diseases like rheumatoid arthritis, septic arthritis and/or avascular necrosis; and/or formation of multi-nucleated chondroclasts; (k) regulatory T cells (Treg): down-regulation of hyper-activated and functionally reversed Tregs which promote inflammation instead of dampening; and/or (l) innate lymphoid cells Type 2: expression of pro-inflammatory cytokines (such as in influenza, RSV infections, allergies and/or asthma), migration and adhesion in pathological settings and/or platelet-ILC2 adhesion in asthma. In an embodiment, the inflammatory processes are neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration and are inhibited by compounds of the present disclosure.

The terms “inhibiting”, “inhibited” and the like as used herein, e.g., in respect to the inflammatory processes described above and/or the conditions, diseases and disorders treatable by inhibiting SWAP-70 and/or treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration, means an inhibition that causes a therapeutic effect and may be any detectable inhibition in the presence of a compound of the present disclosure in comparison to the same conditions without the compound. It will be appreciated by a person skilled in the art having regard to the present disclosure that there are some functions of SWAP-70 such as supporting a proper immune response, which may not be desirable to inhibit—as long as the immune system stays balanced. In contrast, if these functions are excessively activated, down-regulation may be potentially beneficial, and in such cases, such processes are desirably only moderately down-regulated and not entirely abrogated. Such inflammatory processes include, for example, those described above as “excessive”.

The terms “to treat”, “treating” and “treatment” and the like as used herein and as is well understood in the art, means an approach for obtaining beneficial or desired results, including clinical results. For example, in the context of treating lung inflammation, beneficial or desired clinical results include, but are not limited to alleviation or amelioration of one or more symptoms of the lung inflammation, diminishment of the extent of the lung inflammation, stabilized (i.e., not worsening) of the lung inflammation, delay or slowing of the progression of the lung inflammation, amelioration or palliation of the disease state of the lung inflammation, diminishment of the reoccurrence of the lung inflammation, and/or remission (whether partial or total) of the lung inflammation, whether detectable or undetectable. “To treat”, “treating” and “treatment” and the like as used herein also include prophylactic treatment.

The compounds of the disclosure are, for example, administered to the subject or used in an “effective amount”. As used herein, the term “effective amount” and the like means an amount effective, at dosages and for periods of time necessary to achieve a desired result. For example, in the context of treating lung inflammation, an effective amount of a compound administered or used is an amount that, for example, reduces the lung inflammation compared to the lung inflammation without administration or use of the compound. Effective amounts may vary according to factors such as the disease state, age, sex, weight and/or species of the subject. The amount of a given compound that will correspond to such an amount will vary depending upon various factors, such as the given compound, the pharmaceutical formulation, the route of administration or use, the type of condition, disease or disorder being treated, the identity of the subject being treated, and the like, but can nevertheless be routinely determined by one skilled in the art.

### II. Methods of Treatment and Uses

The compound B2 was selected in vitro for inhibition of SWAP-70 and subsequently shown to block SWAP-70 dimerization. B2 has been shown herein to inhibit the corresponding disease-relevant processes in distinct primary human leukocyte populations. Accordingly, B2 and/or other compounds of the present disclosure may be useful to treat diseases, disorders or conditions treatable by inhibiting SWAP-70 and/or treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration and/or for the treatment of inflammatory diseases, disorders or conditions.

Accordingly, the present disclosure includes a method of treating an inflammatory disease, disorder or condition, the method comprising administering an effective amount of a compound of Formula I or a pharmaceutically acceptable salt, solvate and/or prodrug thereof to a subject in need thereof:

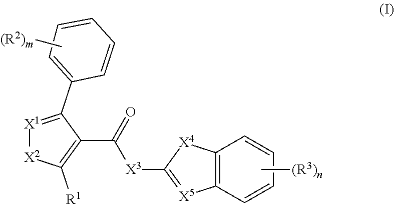

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a use of an effective amount of a compound of Formula I or a pharmaceutically acceptable salt, solvate and/or prodrug thereof for treating an inflammatory disease, disorder or condition in a subject in need thereof:

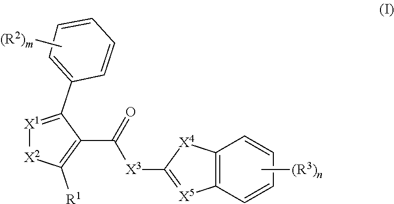

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a use of an effective amount of a compound of Formula I or a pharmaceutically acceptable salt, solvate and/or prodrug thereof for preparation of a medicament for treating an inflammatory disease, disorder or condition in a subject in need thereof:

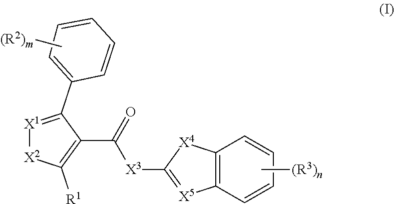

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a compound of Formula I or a pharmaceutically acceptable salt, solvate and/or prodrug thereof for use in the treatment of an inflammatory disease, disorder or condition:

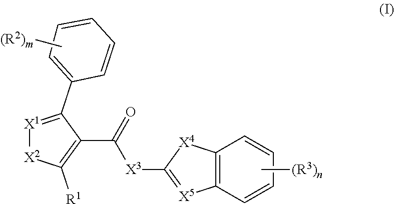

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a method of treating a disease, disorder or condition treatable by inhibiting SWAP-70, the method comprising administering an effective amount of a compound of Formula I or a pharmaceutically acceptable salt, solvate and/or prodrug thereof to a subject in need thereof:

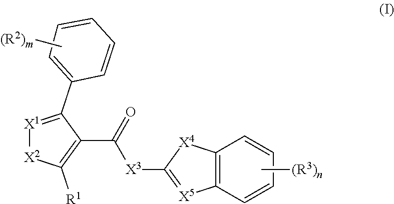

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a use of an effective amount of a compound of Formula I or a pharmaceutically acceptable salt, solvate and/or prodrug thereof for treating a disease, disorder or condition treatable by inhibiting SWAP-70 in a subject in need thereof:

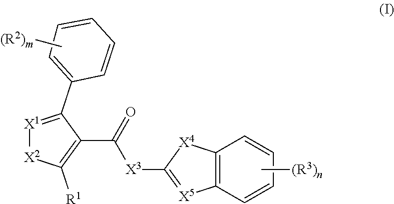

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a use of an effective amount of a compound of Formula I or a pharmaceutically acceptable salt, solvate and/or prodrug thereof for preparation of a medicament for treating a disease, disorder or condition treatable by inhibiting SWAP-70 in a subject in need thereof:

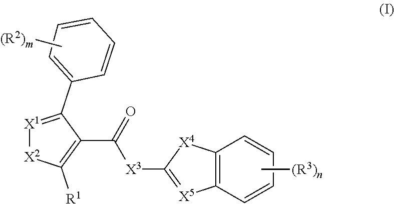

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a compound of Formula I or a pharmaceutically acceptable salt, solvate and/or prodrug thereof for use to treat a disease, disorder or condition treatable by inhibiting SWAP-70 in a subject in need thereof:

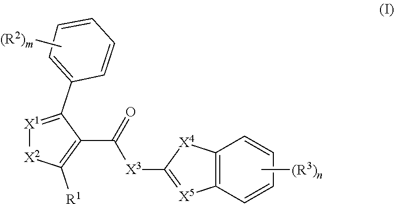

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a method of treating a disease, disorder or condition treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration, the method comprising administering an effective amount of a compound of Formula I or a pharmaceutically acceptable salt, solvate and/or prodrug thereof to a subject in need thereof:

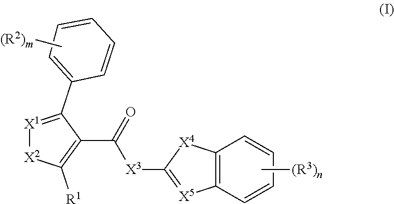

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a use of an effective amount of a compound of Formula I or a pharmaceutically acceptable salt, solvate and/or prodrug thereof for treating a disease, disorder or condition treatable by neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration in a subject in need thereof:

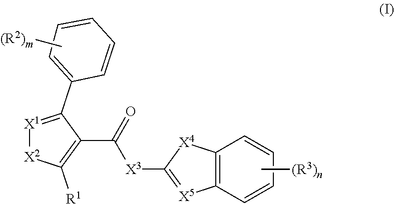

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a use of an effective amount of a compound of Formula I or a pharmaceutically acceptable salt, solvate and/or prodrug thereof for preparation of a medicament for treating a disease, disorder or condition treatable by neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration in a subject in need thereof:

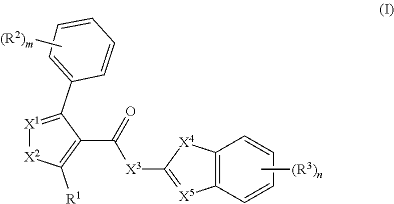

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

The present disclosure also includes a compound of Formula I or a pharmaceutically acceptable salt, solvate and/or prodrug thereof for use to treat a disease, disorder or condition treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration in a subject in need thereof:

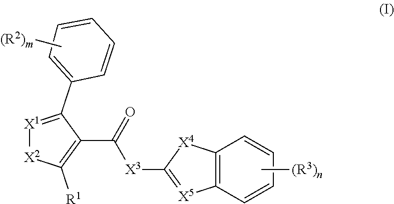

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH;
   R⁴, R⁵, R⁶, R⁷ and R⁸ are each independently selected from H, C₁₋₁₀alkyl, substituted or unsubstituted aryl and heteroaryl;
   m is an integer of from 0 to 5; and
   n is an integer of from 0 to 4.

In an embodiment, m is an integer of from 1 to 5, 1 to 4, 1 to 3 or 1 to 2. In another embodiment, m is 4, 3, 2 or 1. In an embodiment, m is not 0. In an embodiment, m is not 2. In an embodiment, m is 1. In an embodiment, when m is 0, R³ is selected from substituted or unsubstituted aryl, heteroaryl, halo, NO₂ and SO₂R⁵. In another embodiment, m is 1 and R² is halo, n is an integer of from 1 to 4, and R³ is selected from substituted or unsubstituted aryl, heteroaryl and NO₂. In a further embodiment, when m is 2, R² is selected from C₁₋₁₀alkyl, substituted or unsubstituted aryl, heteroaryl, OR⁴, NO₂ and SO₂R⁵.

In an embodiment, n is an integer of from 1 to 4, 1 to 3 or 1 to 2. In another embodiment, n is 3, 2 or 1. In a further embodiment, n is 1.

In an embodiment:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

In an embodiment, the compound of Formula I is a compound of Formula I(a):

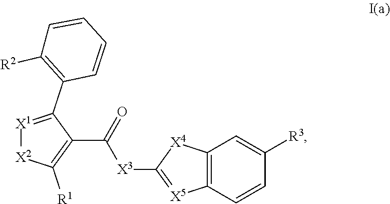

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl; or
   R¹ is selected from H, C₁₋₁₀alkyl, substituted or unsubstituted aryl and heteroaryl;
   R² is selected from C₁₋₁₀alkyl, substituted or unsubstituted aryl, heteroaryl, halo, OR⁴, NO₂ and SO₂R⁵;
   R³ is NO₂;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl.

In an embodiment, R¹ is H. In another embodiment, R¹ is selected from C₁₋₁₀alkyl, substituted or unsubstituted aryl and heteroaryl. In a further embodiment, R¹ is C₁₋₁₀alkyl. In another embodiment, R¹ is C₁₋₅alkyl. In a further embodiment, R¹ is C₁₋₄alkyl. In another embodiment, R¹ is methyl.

In an embodiment, R² is halo. In another embodiment, R² is chloro.

In an embodiment, R³ is NO₂. In an embodiment, R³ is not SO₂R⁵.

In an embodiment, R⁴ is H. In another embodiment, R⁴ is selected from C₁₋₁₀alkyl, substituted or unsubstituted aryl and heteroaryl. In a further embodiment, R⁴ is C₁₋₁₀alkyl. In another embodiment, R⁴ is substituted or unsubstituted aryl. In an embodiment, R⁴ is heteroaryl.

In an embodiment, R⁵ is H. In another embodiment, R⁵ is selected from C₁₋₁₀alkyl, substituted or unsubstituted aryl and heteroaryl. In a further embodiment, R⁵ is C₁₋₁₀alkyl. In another embodiment, R⁵ is substituted or unsubstituted aryl. In an embodiment, R⁵ is heteroaryl.

In an embodiment, R⁶ is H. In another embodiment, R⁶ is selected from C₁₋₁₀alkyl, substituted or unsubstituted aryl and heteroaryl. In a further embodiment, R⁶ is C₁₋₁₀alkyl. In another embodiment, R⁶ is substituted or unsubstituted aryl. In an embodiment, R⁶ is heteroaryl.

In an embodiment, R⁷ is H. In another embodiment, R⁷ is selected from C₁₋₁₀alkyl, substituted or unsubstituted aryl and heteroaryl. In a further embodiment, R⁷ is C₁₋₁₀alkyl. In another embodiment, R⁷ is substituted or unsubstituted aryl. In an embodiment, R⁷ is heteroaryl.

In an embodiment, R⁸ is H. In another embodiment, R⁸ is selected from C₁₋₁₀alkyl, substituted or unsubstituted aryl and heteroaryl. In a further embodiment, R⁸ is C₁₋₁₀alkyl. In another embodiment, R⁸ is substituted or unsubstituted aryl. In an embodiment, R⁸ is heteroaryl.

In an embodiment, X¹ is N.

In an embodiment, X² is O.

In an embodiment, X³ is NR⁷. In another embodiment, X³ is NH.

In an embodiment, X⁴ is S.

In an embodiment, X⁵ is N.

In an embodiment, X¹ is N, X² is O, X³ is NH, X⁴ is S and/or X⁵ is N.

In an embodiment, the compound of Formula I has the structure:

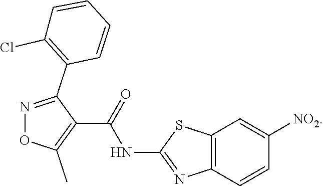 [SMILES: Cc1onc(-c2ccccc2Cl)c1C(=O)Nc1nc2ccc([N+](=O)[O-])cc2s1]

In an embodiment, the compound of Formula I is a compound of Formula I(b) or a pharmaceutically acceptable salt, solvate and/or prodrug thereof:

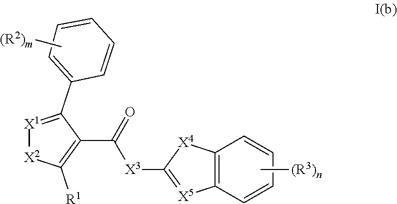

wherein
   m is an integer of from 0 to 5;
   n is an integer of from 0 to 4;
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl;
provided that:
   when m is 0, R³ is selected from substituted or unsubstituted aryl, heteroaryl, halo, NO₂ and SO₂R⁵;
   when m is 1 and R² is halo, n is an integer of from 1 to 4, and R³ is selected from substituted or unsubstituted aryl, heteroaryl and NO₂; and
   when m is 2, R² is selected from C₁₋₁₀alkyl, substituted or unsubstituted aryl, heteroaryl, OR⁴, NO₂ and SO₂R⁵.

In an embodiment, m is an integer of from 1 to 5, 1 to 4, 1 to 3 or 1 to 2. In another embodiment, m is 4, 3, 2 or 1. In an embodiment, m is not 0. In an embodiment, m is not 2. In an embodiment, m is 1.

In an embodiment, n is an integer of from 1 to 4, 1 to 3 or 1 to 2. In another embodiment, n is 3, 2 or 1. In a further embodiment, n is 1.

In an embodiment:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

In an embodiment, the compound of Formula I(b) is a compound of Formula I(c):

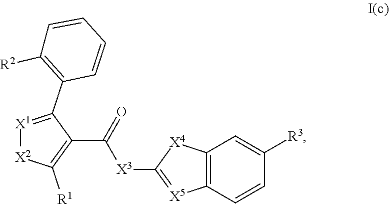

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl;
provided that:
   when m is 0, R³ is selected from substituted or unsubstituted aryl, heteroaryl, halo, NO₂ and SO₂R⁵;
   when m is 1 and R² is halo, n is an integer of from 1 to 4, and R³ is selected from substituted or unsubstituted aryl, heteroaryl and NO₂; and
   when m is 2, R² is selected from C₁₋₁₀alkyl, substituted or unsubstituted aryl, heteroaryl, OR⁴, NO₂ and SO₂R⁵; or
   R¹ is selected from H, C₁₋₁₀alkyl, substituted or unsubstituted aryl and heteroaryl;
   R² is selected from C₁₋₁₀alkyl, substituted or unsubstituted aryl, heteroaryl, halo, OR⁴, NO₂ and SO₂R⁵;
   R³ is NO₂;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl.

In an embodiment, R¹ is H. In another embodiment, R¹ is selected from C₁₋₁₀alkyl, substituted or unsubstituted aryl and heteroaryl. In a further embodiment, R¹ is C₁₋₁₀alkyl. In another embodiment, R¹ is C₁₋₅alkyl. In a further embodiment, R¹ is C₁₋₄alkyl. In another embodiment, R¹ is methyl.

In an embodiment, R² is halo. In another embodiment, R² is chloro.

In an embodiment, R³ is NO₂. In an embodiment, R³ is not SO₂R⁵.

In an embodiment, R⁴ is H. In another embodiment, R⁴ is selected from C₁₋₁₀alkyl, substituted or unsubstituted aryl and heteroaryl. In a further embodiment, R⁴ is C₁₋₁₀alkyl. In another embodiment, R⁴ is substituted or unsubstituted aryl. In an embodiment, R⁴ is heteroaryl.

In an embodiment, R⁵ is H. In another embodiment, R⁵ is selected from C₁₋₁₀alkyl, substituted or unsubstituted aryl and heteroaryl. In a further embodiment, R⁵ is C₁₋₁₀alkyl. In another embodiment, R⁵ is substituted or unsubstituted aryl. In an embodiment, R⁵ is heteroaryl.

In an embodiment, R⁶ is H. In another embodiment, R⁶ is selected from C₁₋₁₀alkyl, substituted or unsubstituted aryl and heteroaryl. In a further embodiment, R⁶ is C₁₋₁₀alkyl. In another embodiment, R⁶ is substituted or unsubstituted aryl. In an embodiment, R⁶ is heteroaryl.

In an embodiment, R⁷ is H. In another embodiment, R⁷ is selected from C₁₋₁₀alkyl, substituted or unsubstituted aryl and heteroaryl. In a further embodiment, R⁷ is C₁₋₁₀alkyl. In another embodiment, R⁷ is substituted or unsubstituted aryl. In an embodiment, R⁷ is heteroaryl.

In an embodiment, R⁸ is H. In another embodiment, R⁸ is selected from C₁₋₁₀alkyl, substituted or unsubstituted aryl and heteroaryl. In a further embodiment, R⁸ is C₁₋₁₀alkyl. In another embodiment, R⁸ is substituted or unsubstituted aryl. In an embodiment, R⁸ is heteroaryl.

In an embodiment, X¹ is N.

In an embodiment, X² is O.

In an embodiment, X³ is NR⁷. In another embodiment, X³ is NH.

In an embodiment, X⁴ is S.

In an embodiment, X⁵ is N.

In an embodiment, R¹ is C₁₋₅alkyl; R² is halo; R³ is NO₂; X¹ is N; X² is O; X³ is NH; X⁴ is S; X⁵ is N; m is 1; and n is 1.

The present disclosure also includes a compound of Formula I(b) or a pharmaceutically acceptable salt, solvate and/or prodrug thereof for use as a medicament:

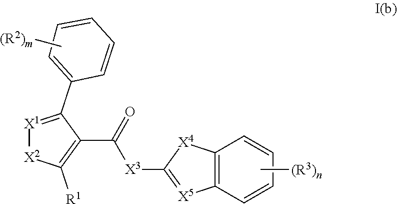

wherein
   m is an integer of from 0 to 5;
   n is an integer of from 0 to 4;
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl;
provided that:
   when m is 0, R³ is selected from substituted or unsubstituted aryl, heteroaryl, halo, NO₂ and SO₂R⁵;
   when m is 1 and R² is halo, n is an integer of from 1 to 4, and R³ is selected from substituted or unsubstituted aryl, heteroaryl and NO₂; and
   when m is 2, R² is selected from C₁₋₁₀alkyl, substituted or unsubstituted aryl, heteroaryl, OR⁴, NO₂ and SO₂R⁵.

In an embodiment, m is an integer of from 1 to 5, 1 to 4, 1 to 3 or 1 to 2. In another embodiment, m is 4, 3, 2 or 1. In an embodiment, m is not 0. In an embodiment, m is not 2. In an embodiment, m is 1.

In an embodiment, n is an integer of from 1 to 4, 1 to 3 or 1 to 2. In another embodiment, n is 3, 2 or 1. In a further embodiment, n is 1.

In an embodiment:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

In an embodiment, the compound of Formula I(b) is a compound of Formula I(c):

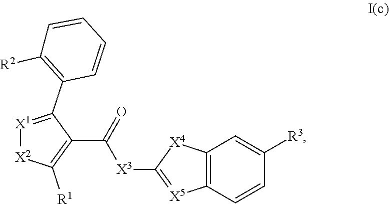

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl;
provided that:
   when m is 0, R³ is selected from substituted or unsubstituted aryl, heteroaryl, halo, NO₂ and SO₂R⁵;
   when m is 1 and R² is halo, n is an integer of from 1 to 4, and R³ is selected from substituted or unsubstituted aryl, heteroaryl and NO₂; and
   when m is 2, R² is selected from C₁₋₁₀alkyl, substituted or unsubstituted aryl, heteroaryl, OR⁴, NO₂ and SO₂R⁵; or
   R¹ is selected from H, C₁₋₁₀alkyl, substituted or unsubstituted aryl and heteroaryl;
   R² is selected from C₁₋₁₀alkyl, substituted or unsubstituted aryl, heteroaryl, halo, OR⁴, NO₂ and SO₂R⁵;
   R³ is NO₂;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl.

In an embodiment, R¹ is H. In another embodiment, R¹ is selected from C₁₋₁₀alkyl, substituted or unsubstituted aryl and heteroaryl. In a further embodiment, R¹ is C₁₋₁₀alkyl. In another embodiment, R¹ is C₁₋₅alkyl. In a further embodiment, R¹ is C₁₋₄alkyl. In another embodiment, R¹ is methyl.

In an embodiment, R² is halo. In another embodiment, R² is chloro.

In an embodiment, R³ is NO₂. In an embodiment, R³ is not SO₂R⁵.

In an embodiment, R⁴ is H. In another embodiment, R⁴ is selected from C₁₋₁₀alkyl, substituted or unsubstituted aryl and heteroaryl. In a further embodiment, R⁴ is C₁₋₁₀alkyl. In another embodiment, R⁴ is substituted or unsubstituted aryl. In an embodiment, R⁴ is heteroaryl.

In an embodiment, R⁵ is H. In another embodiment, R⁵ is selected from C₁₋₁₀alkyl, substituted or unsubstituted aryl and heteroaryl. In a further embodiment, R⁵ is C₁₋₁₀alkyl. In another embodiment, R⁵ is substituted or unsubstituted aryl. In an embodiment, R⁵ is heteroaryl.

In an embodiment, R⁶ is H. In another embodiment, R⁶ is selected from C₁₋₁₀alkyl, substituted or unsubstituted aryl and heteroaryl. In a further embodiment, R⁶ is C₁₋₁₀alkyl. In another embodiment, R⁶ is substituted or unsubstituted aryl. In an embodiment, R⁶ is heteroaryl.

In an embodiment, R⁷ is H. In another embodiment, R⁷ is selected from C₁₋₁₀alkyl, substituted or unsubstituted aryl and heteroaryl. In a further embodiment, R⁷ is C₁₋₁₀alkyl. In another embodiment, R⁷ is substituted or unsubstituted aryl. In an embodiment, R⁷ is heteroaryl.

In an embodiment, R⁸ is H. In another embodiment, R⁸ is selected from C₁₋₁₀alkyl, substituted or unsubstituted aryl and heteroaryl. In a further embodiment, R⁸ is C₁₋₁₀alkyl. In another embodiment, R⁸ is substituted or unsubstituted aryl. In an embodiment, R⁸ is heteroaryl.

In an embodiment, X¹ is N.

In an embodiment, X² is O.

In an embodiment, X³ is NR⁷. In another embodiment, X³ is NH.

In an embodiment, X⁴ is S.

In an embodiment, X⁵ is N.

In an embodiment, R¹ is C₁₋₅alkyl; R² is halo; R³ is NO₂; X¹ is N; X² is O; X³ is NH; X⁴ is S; X⁵ is N; m is 1; and n is 1.

In an embodiment, the compound of Formula I has the structure:

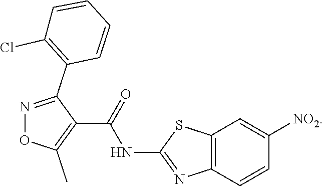 [SMILES: Cc1onc(-c2ccccc2Cl)c1C(=O)Nc1nc2ccc([N+](=O)[O-])cc2s1]

In an embodiment, the compound of Formula I or the pharmaceutically acceptable salt, solvate and/or prodrug thereof is the compound of Formula I; i.e., is not the pharmaceutically acceptable salt, solvate and/or prodrug of the compound of Formula I.

In an embodiment, the subject is a human.

The disease, disorder or condition can be any suitable inflammatory disease, disorder or condition and/or any suitable disease, disorder or condition treatable by inhibiting SWAP-70 and/or treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration.

In an embodiment, the disease, disorder or condition is treatable by inhibiting neutrophil reactive oxygen species (ROS) production. In another embodiment, the disease, disorder or condition is treatable by inhibiting neutrophil NETosis. In another embodiment, the disease, disorder or condition is treatable by inhibiting B lymphocyte plasmablast development. In another embodiment, the disease, disorder or condition is treatable by inhibiting B lymphocyte IgE production. In another embodiment, the disease, disorder or condition is treatable by inhibiting mast cell degranulation. In another embodiment, the disease, disorder or condition is treatable by inhibiting eosinophil reactive oxygen species (ROS) production. In another embodiment, the disease, disorder or condition is treatable by inhibiting mast cell cytokine release. In another embodiment, the disease, disorder or condition is treatable by inhibiting B lymphocyte cytokine release. In another embodiment, the disease, disorder or condition is treatable by inhibiting endothelial cell cytokine release. In another embodiment, the disease, disorder or condition is treatable by inhibiting endothelial cell-immune cell interactions. In another embodiment, the disease, disorder or condition is treatable by inhibiting macrophage/monocyte migration. In another embodiment, the disease, disorder or condition is treatable by a combination of inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and macrophage/monocyte migration.

In an embodiment, the disease, disorder or condition is a microbial infection, asthma, systemic inflammation, endothelial inflammation, an allergy, urticaria, osteoporosis, chronic obstructive pulmonary disease (COPD), arthritis, an autoimmune disorder, a cytokine release syndrome, avascular necrosis and/or sepsis.

In an embodiment, the disease, disorder or condition is a microbial infection. In an embodiment, the microbial infection is a viral infection or a bacterial infection. In a further embodiment, the microbial infection is a viral infection. In an embodiment, the viral infection is caused by SARS-CoV-2 virus, respiratory syncytial virus (RSV) or an influenza virus (e.g., influenza A). In another embodiment, the viral infection is caused by SARS-CoV-2 virus. Accordingly, in an embodiment, the compound of Formula I or the pharmaceutically acceptable salt thereof is for treatment of disease caused by the SARS-CoV-2 virus. In a further embodiment, the microbial infection is a bacterial infection. In an embodiment, the bacteria is Yersinia pestis.

In an embodiment, the treatment is for lung inflammation. The lung inflammation can be virus-induced lung inflammation or non-viral lung inflammation. In an embodiment, the lung inflammation is virus-induced lung inflammation. In another embodiment, the lung inflammation is bacteria-induced lung inflammation. For example, in an embodiment, the microbial infection is a viral infection, and the treatment is for virus-induced lung inflammation. In an embodiment, treatment of the lung inflammation prevents subsequent opportunistic pneumonia. In another embodiment, the opportunistic pneumonia is caused by a virus (e.g., rhinovirus or influenza), bacteria (e.g., Streptococcus pneumoniae) or combinations thereof.

In an embodiment, the disease, disorder or condition is systemic inflammation. The systemic inflammation can be virus-induced systemic inflammation or non-viral systemic inflammation. The systemic inflammation can, for example, be as a consequence of lung inflammation or as a direct inflammatory process. In an embodiment, the systemic inflammation comprises inflammation of the gastrointestinal system, kidneys, heart, the nervous system, the skin and/or liver.

In an embodiment, the disease, disorder or condition is endothelial inflammation. In another embodiment, the treatment is for atherosclerosis and/or coronary artery disease.

In an embodiment, the disease, disorder or condition is sepsis. In another embodiment, the sepsis is bacterial sepsis.

In an embodiment, the disease, disorder or condition is allergy. In another embodiment, the allergy is an IgE-mediated allergy selected from a contact sensitivity, atopic dermatitis, a food allergy (such as a nut allergy), hay fever and a venom allergy.

In an embodiment, the disease, disorder or condition is asthma. In an embodiment, the asthma is triggered by an IgE-allergen reaction. In another embodiment, the asthma is chronic asthma. In another embodiment, the asthma is acute asthma. In a further embodiment, the treatment is for prevention of acute asthma episodes.

In an embodiment, the disease, disorder or condition is urticaria. In an embodiment, the urticaria is pseudo-allergic urticaria. In another embodiment, the urticaria is acute. In a further embodiment, the urticaria is chronic.

In an embodiment, the disease, disorder or condition is osteoporosis.

In an embodiment, the disease, disorder or condition is chronic obstructive pulmonary disease (COPD). In an embodiment, the treatment is for an acute COPD episode. In another embodiment, the treatment is for chronic COPD. In another embodiment, the treatment is for prevention of progressive COPD disease in a subject with chronic COPD.

In an embodiment, the disease, disorder or condition is arthritis (e.g., inflammatory arthritis). In an embodiment, the arthritis is septic arthritis and/or rheumatoid arthritis (RA). In another embodiment, the arthritis is rheumatoid arthritis (RA). In a further embodiment, the treatment is for an acute episode of RA. In another embodiment, the treatment is for the prevention of worsening RA. In another embodiment, the inflammatory arthritis is gout.

In an embodiment, the disease, disorder or condition is an auto-immune disorder. In another embodiment, the auto-immune disorder is rheumatoid arthritis, systemic lupus erythematosus, bullous pemphigoid, psoriasis or atopic dermatitis.

In an embodiment, the disease, disorder or condition is a cytokine release syndrome. In an embodiment, the treatment is for thrombosis associated with the cytokine storm. In an embodiment, the thrombosis is associated with soluble adhesion molecules inhibited by a compound of the present disclosure. In an embodiment, the treatment is for venous thromboembolism. In another embodiment, the treatment is for inflammatory arterial thrombosis.

In an embodiment, the disease, disorder or condition is avascular necrosis.

In an embodiment, the subject has a combination of two or more diseases, disorders or conditions selected from a microbial infection, asthma, systemic inflammation, endothelial inflammation, an allergy, urticaria, osteoporosis, chronic obstructive pulmonary disease (COPD), arthritis, an autoimmune disorder, a cytokine release syndrome, avascular necrosis and sepsis. For example, in an embodiment, the treatment is of a subject having a non-infectious pulmonary condition (e.g., asthma or COPD) which is exacerbated by a viral infection (such as but not limited to RSV, influenza, rhinovirus, parainfluenza, and/or coronaviruses). In an embodiment, the subject to be treated has respiratory distress syndrome (ARDS) which was caused by the viral infection.

The present disclosure also includes a composition comprising one or more compounds of the present disclosure and optionally a carrier. The compounds of the disclosure are optionally formulated into pharmaceutical compositions for administration to subjects or use in a biologically compatible form suitable for administration or use in vivo. Accordingly, the present disclosure further includes a pharmaceutical composition comprising one or more compounds of the present disclosure and optionally a pharmaceutically acceptable carrier. In another embodiment, the pharmaceutical composition comprises the pharmaceutically acceptable carrier. In another embodiment of the present disclosure, the pharmaceutical composition comprises a pharmaceutically acceptable thinner or carrier, and at least one further component selected from the group consisting of diluents, fillers, binders, and other excipients or stabilizers.

In an embodiment, the pharmaceutical composition comprises a compound of Formula I(b) or a pharmaceutically acceptable salt, solvate and/or prodrug thereof:

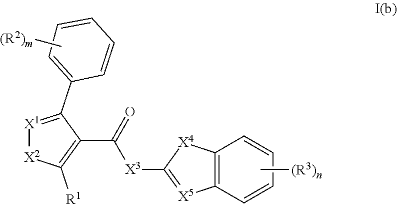

wherein
   m is an integer of from 0 to 5;
   n is an integer of from 0 to 4;
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl;
provided that:
   when m is 0, R³ is selected from substituted or unsubstituted aryl, heteroaryl, halo, NO₂ and SO₂R⁵;
   when m is 1 and R² is halo, n is an integer of from 1 to 4, and R³ is selected from substituted or unsubstituted aryl, heteroaryl and NO₂; and
   when m is 2, R² is selected from C₁₋₁₀alkyl, substituted or unsubstituted aryl, heteroaryl, OR⁴, NO₂ and SO₂R⁵.

In an embodiment, m is an integer of from 1 to 5, 1 to 4, 1 to 3 or 1 to 2. In another embodiment, m is 4, 3, 2 or 1. In an embodiment, m is not 0. In an embodiment, m is not 2. In an embodiment, m is 1.

In an embodiment, n is an integer of from 1 to 4, 1 to 3 or 1 to 2. In another embodiment, n is 3, 2 or 1. In a further embodiment, n is 1.

In an embodiment:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

In an embodiment, the compound of Formula I(b) is a compound of Formula I(c):

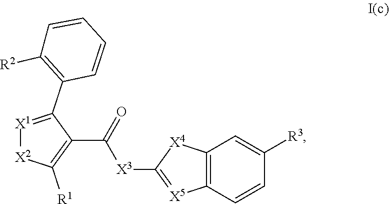

wherein
   R¹ is selected from H, C₁₋₁₀alkyl, substituted or unsubstituted aryl and heteroaryl;
   R² and R³ are each independently selected from C₁₋₁₀alkyl, substituted or unsubstituted aryl, heteroaryl, halo, OR⁴, NO₂ and SO₂R⁵;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl;
provided that:
   when m is 0, R³ is selected from substituted or unsubstituted aryl, heteroaryl, halo, NO₂ and SO₂R⁵;
   when m is 1 and R² is halo, n is an integer of from 1 to 4, and R³ is selected from substituted or unsubstituted aryl, heteroaryl and NO₂; and
   when m is 2, R² is selected from C₁₋₁₀alkyl, substituted or unsubstituted aryl, heteroaryl, OR⁴, NO₂ and SO₂R⁵; or
   R¹ is selected from H, C₁₋₁₀alkyl, substituted or unsubstituted aryl and heteroaryl;
   R² is selected from C₁₋₁₀alkyl, substituted or unsubstituted aryl, heteroaryl, halo, OR⁴, NO₂ and SO₂R⁵;
   R³ is NO₂;
   X¹ is selected from N and CH;
   X² is selected from O, S and NR⁶;
   X³ is selected from O and NR⁷;
   X⁴ is selected from O, S, CH₂, SO, SO₂ and NR⁸;
   X⁵ is selected from N and CH; and
   R⁴, R⁵, R⁶, R⁷ and R⁸ are each independently selected from H, C₁₋₁₀alkyl, substituted or unsubstituted aryl and heteroaryl.

In an embodiment, R¹ is H. In another embodiment, R¹ is selected from C₁₋₁₀alkyl, substituted or unsubstituted aryl and heteroaryl. In a further embodiment, R¹ is C₁₋₁₀alkyl. In another embodiment, R¹ is C₁₋₅alkyl. In a further embodiment, R¹ is C₁₋₄alkyl. In another embodiment, R¹ is methyl.

In an embodiment, R² is halo. In another embodiment, R² is chloro.

In an embodiment, R³ is NO₂. In an embodiment, R³ is not SO₂R⁵.

In an embodiment, R⁴ is H. In another embodiment, R⁴ is selected from C₁₋₁₀alkyl, substituted or unsubstituted aryl and heteroaryl. In a further embodiment, R⁴ is C₁₋₁₀alkyl. In another embodiment, R⁴ is substituted or unsubstituted aryl. In an embodiment, R⁴ is heteroaryl.

In an embodiment, R⁵ is H. In another embodiment, R⁵ is selected from C₁₋₁₀alkyl, substituted or unsubstituted aryl and heteroaryl. In a further embodiment, R⁵ is C₁₋₁₀alkyl. In another embodiment, R⁵ is substituted or unsubstituted aryl. In an embodiment, R⁵ is heteroaryl.

In an embodiment, R⁶ is H. In another embodiment, R⁶ is selected from C₁₋₁₀alkyl, substituted or unsubstituted aryl and heteroaryl. In a further embodiment, R⁶ is C₁₋₁₀alkyl. In another embodiment, R⁶ is substituted or unsubstituted aryl. In an embodiment, R⁶ is heteroaryl.

In an embodiment, R⁷ is H. In another embodiment, R⁷ is selected from C₁₋₁₀alkyl, substituted or unsubstituted aryl and heteroaryl. In a further embodiment, R⁷ is C₁₋₁₀alkyl. In another embodiment, R⁷ is substituted or unsubstituted aryl. In an embodiment, R⁷ is heteroaryl.

In an embodiment, R⁸ is H. In another embodiment, R⁸ is selected from C₁₋₁₀alkyl, substituted or unsubstituted aryl and heteroaryl. In a further embodiment, R⁸ is C₁₋₁₀alkyl. In another embodiment, R⁸ is substituted or unsubstituted aryl. In an embodiment, R⁸ is heteroaryl.

In an embodiment, X¹ is N.

In an embodiment, X² is O.

In an embodiment, X³ is NR⁷. In another embodiment, X³ is NH.

In an embodiment, X⁴ is S.

In an embodiment, X⁵ is N.

In an embodiment, R¹ is C₁₋₅alkyl; R² is halo; R³ is NO₂; X¹ is N; X² is O; X³ is NH; X⁴ is S; X⁵ is N; m is 1; and n is 1.

In an embodiment, the compound of Formula I(b) has the structure:

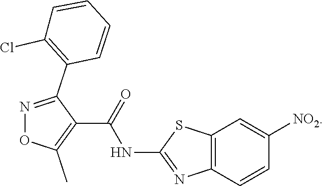 [SMILES: Cc1onc(-c2ccccc2Cl)c1C(=O)Nc1nc2ccc([N+](=O)[O-])cc2s1]

In an embodiment, the compound of Formula I(b) or the pharmaceutically acceptable salt, solvate and/or prodrug thereof is the compound of Formula I(b); i.e., is not the pharmaceutically acceptable salt, solvate and/or prodrug of the compound.

The present disclosure also includes a pharmaceutical composition as described herein for use in any of the methods of treating or uses as described herein. For example, the present disclosure includes a method of treating an inflammatory disease, disorder or condition as described herein, comprising administering an effective amount of a pharmaceutical composition as described herein to a subject in need thereof; a method of treating a disease, disorder or condition treatable by inhibiting SWAP-70 as described herein, the method comprising administering an effective amount of a pharmaceutical composition as described herein to a subject in need thereof; and a method of treating a disease, disorder or condition treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration as described herein, the method comprising administering an effective amount of a pharmaceutical composition as described herein to a subject in need thereof as well as the corresponding uses. It will be appreciated by a person skilled in the art that the embodiments of such methods and uses can be varied as described herein in respect to the methods and uses in respect to the compounds of the present disclosure described hereinabove.

The compounds of the disclosure can be administered to a subject or used in a variety of forms depending on the selected route of administration or use, as will be understood by those skilled in the art. In an embodiment, the one or more compounds of the disclosure are administered to the subject, or used, by oral (peroral) or parenteral (including injection (e.g., intravenous (i.v.), intramuscular (i.m.), intraperitoneal (i.p.), subcutaneous (s.c.) and intrathecal (i.t.) forms of administration or use), infusion, via implant, inhalational, intranasal, sublingual, buccal, rectal, vaginal, topical, transdermal, ocular and otic administration or use and the compound(s) formulated accordingly. For example, the compounds of the disclosure are administered or used in an injection, in a spray, in a tablet/caplet, in a powder, topically, in a gel, in drops, by a patch, by an implant, by a slow-release pump or by any other suitable method of administration or use, the selection of which can be made by a person skilled in the art.

In an embodiment, the one or more compounds of the present disclosure are orally administered or used, for example, with an inert diluent or with an assimilable edible carrier, or enclosed in hard or soft shell gelatin capsules, or compressed into tablets, or incorporated directly with the food of the diet. In an embodiment, for oral therapeutic administration or use, the one or more compounds of the disclosure are incorporated with excipient and administered or used in the form of ingestible tablets, buccal tablets, troches, capsules, elixirs, suspensions, syrups, wafers, and the like. Oral dosage forms also include modified release, for example immediate release and timed-release, formulations. In another embodiment of the present disclosure, the one or more compounds of the present disclosure are administered or used parenterally. Pharmaceutical forms suitable for injectable administration or use include sterile aqueous solutions or dispersions and sterile powders for the extemporaneous preparation of sterile injectable solutions or dispersions. A person skilled in the art would know how to prepare suitable formulations. In an embodiment, the the one or more compounds of the present disclosure are administered via inhalation.

Treatment methods or uses comprise administering to a subject or use of an effective amount of one or more compounds of the disclosure, optionally consisting of a single administration or use, or alternatively comprising a series of administrations or uses. For example, the compounds of the disclosure are administered or used at least once a week. However, in another embodiment, the compounds are administered to the subject or used from one time per three weeks, or one time per week to once daily for a given treatment or use. In another embodiment, the compounds are administered or used 2, 3, 4, 5 or 6 times daily. The length of the treatment period or use depends on a variety of factors, such as the identity of the disease, disorder or condition, the severity of the disease, disorder or condition, the age of the subject, the concentration of the one or more compounds in a formulation, the activity of the compounds of the present disclosure, and/or a combination thereof. It will also be appreciated that the effective amount of a compound used for the treatment or use may increase or decrease over the course of a particular treatment regime or use. Changes in dosage may result and become apparent by standard diagnostic assays known in the art. In some instances, chronic administration or use is required. For example, the one or more compounds of the present disclosure are administered or used in an amount and for duration sufficient to treat the subject.

The extent and/or undesirable clinical manifestations of the disease, disorder or condition are optionally lessened (palliated) and/or the time course of the progression is slowed or lengthened, as compared to not treating the disease, disorder or condition.

The one or more compounds of the disclosure may be administered or used alone or in combination with other therapeutic agents useful for treating the disease, disorder or condition. When administered or used in combination with other known therapeutic agents, it is an embodiment that the one or more compounds of the disclosure are administered or used contemporaneously with those therapeutic agents. As used herein the term “contemporaneous” in reference to administration of two substances to a subject or use means providing each of the two substances so that they are both biologically active in the individual at the same time. The exact details of the administration or use will depend on the pharmacokinetics of the two substances in the presence of each other and can include administering or using the two substances within a few hours of each other, or even administering or using one substance within 24 hours of administration or use of the other, if the pharmacokinetics are suitable. Design of suitable dosing regimens is routine for one skilled in the art. In particular embodiments, two substances will be administered or used substantially simultaneously, i.e., within minutes of each other, or in a single composition that contains both substances. It is a further embodiment that a combination of the two substances is administered to a subject or used in a non-contemporaneous fashion.

The dosage of compounds of the disclosure can vary depending on many factors such as the pharmacodynamic properties of the compound, the mode of administration or use, the age, health and weight of the subject, the identity of the disease, disorder or condition, the nature and extent of the symptoms of the disease, disorder or condition, the frequency of the treatment or use and the type of concurrent treatment or use, if any, and the clearance rate of the compound in the subject. One of skill in the art can determine the appropriate dosage based on the above factors. In an embodiment, the compounds of the disclosure are administered or used initially in a suitable dosage that is optionally adjusted as required, depending on the clinical response. As a representative example, oral dosages of one or more compounds of the disclosure will range from less than 1 mg per day to 1000 mg per day for a human adult or an animal. In an embodiment, the compounds of the disclosure are administered or used in a single daily dose or the total daily dose may be divided into two, three or four daily doses.

### III. Preparation of Compounds of the Present Disclosure

The compounds of the present disclosure can be prepared by various methods, the selection of which can be readily made by a person skilled in the art. Suitable techniques for work-up and/or purification of intermediates and final products can also be selected by the skilled person. Compounds used in the synthesis of the compounds may be available from commercial sources such as but not limited to Sigma-Aldrich, Acros Organics, Lach-Ner or TCI or alternatively may be prepared by a suitable method know to those skilled in the art.

For example, in an embodiment wherein in the compounds of the present disclosure, X¹ is N, X² is O and X³ is NH, the compounds are generally prepared according to a method illustrated in Scheme 1. Variables in Scheme 1 and the following embodiments of the exemplary method for preparation are as defined herein for the compounds of the present disclosure unless otherwise specified and A⁺ is a suitable alkali metal ion such as sodium ion (Na⁺). A person skilled in the art could readily vary the method to prepare, for example, compounds having X¹, X² and/or X³ as otherwise defined herein for the compounds of the present disclosure.

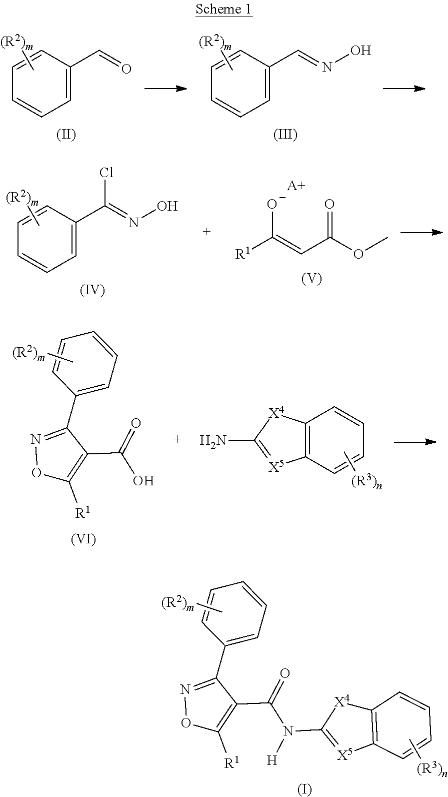

In an embodiment, as shown in Scheme 1, the method comprises reaction of an optionally substituted benzaldehyde of Formula II with a suitable reagent to prepare the oxime of Formula III followed by chlorination of the oxime of Formula III to prepare the chloro-oxime of Formula IV, cyclization of the chloro-oxime of Formula IV and a suitable alkali metal salt (e.g., a sodium salt) of an acetoacetate of Formula V followed by hydrolysis of the methyl ester to prepare the carboxylic acid of Formula VI, which is amide-coupled with a compound of Formula VII (e.g., a 2-amino-benzothiazole derivative) to prepare the compound of Formula I.

In an embodiment, the reaction of the optionally substituted benzaldehyde of Formula II with a suitable reagent to prepare the oxime of Formula III comprises reacting the optionally substituted benzaldehyde of Formula II with hydroxylamine hydrochloride in the presence of a suitable base such as sodium hydroxide in a suitable solvent or mixture thereof such as a mixture of water and ethanol for a time and at a temperature for the conversion of the optionally substituted benzaldehyde of Formula II to the oxime of Formula III to proceed to a sufficient extent.

In an embodiment, the chlorination of the oxime of Formula III to the chloro-oxime of Formula IV comprises reaction of the oxime of Formula III with a suitable chlorinating reagent (e.g., N-chlorosuccinimide) in a suitable solvent or mixture thereof such as N,N-dimethylformamide (DMF) for a time and at a temperature for the conversion of the oxime of Formula III to the chloro-oxime of Formula IV to proceed to a sufficient extent. In an embodiment, the N-chlorosuccinimide is added to a solution of the oxime of Formula III in the suitable solvent or mixture thereof such as DMF portion-wise, for example, in 10, 9, 8, 7, 6, 5, 4, 3 or 2 portions. In another embodiment, the reaction comprises induction with HCl(g).

In an embodiment, the cyclization of the chloro-oxime of Formula IV and the suitable alkali metal salt of an acetoacetate of Formula V comprises reaction of the chloro-oxime of Formula IV and the suitable alkali metal salt of the acetoacetate of Formula V in the presence of a suitable base (e.g., NaOH) in a suitable solvent or mixture thereof such as methanol for a time and at a temperature for the conversion of the chloro-oxime of Formula IV and the suitable alkali metal salt of an acetoacetate of Formula V to the methyl ester of the carboxylic acid of Formula VI to proceed to a sufficient extent. In an embodiment, the alkali metal salt of the acetoacetate of Formula V is a sodium salt. In an embodiment, the suitable alkali metal salt of the acetoacetate of Formula V is added to a solution of the chloro-oxime of Formula IV in the suitable solvent or mixture thereof such as methanol portion-wise, for example, in 15, 14, 13, 12, 11, 10, 9, 8, 7, 6, 5, 4, 3 or 2 portions. In an embodiment, the hydrolysis comprises reaction of the methyl ester of the carboxylic acid of Formula VI with a suitable hydrolyzing agent e.g., a solution of a suitable base such as NaOH in water for a time and at a temperature for the conversion of the methyl ester of the carboxylic acid of Formula VI to the carboxylic acid of Formula VI to proceed to a sufficient extent. In an embodiment, the hydrolysis is carried out in situ; e.g., without work-up, isolation and/or purification of the methyl ester of the carboxylic acid of Formula VI prior to the hydrolysis.

In an embodiment, the amide coupling of the carboxylic acid of Formula VI with the compound of Formula VII (e.g., the 2-amino-benzothiazole derivative) comprises addition of triethylamine and diisopropylcarbodiimide (DIC) to a mixture of the carboxylic acid of Formula VI, the compound of Formula VII and ethyl cyanohydroxyiminoacetate (Oxyma), in a suitable solvent or mixture thereof such as dimethylformamide and reacting for a time and at a temperature for the reaction of the carboxylic acid of Formula VI with the compound of Formula VII to proceed to a sufficient extent, followed by a suitable work-up.

The following are non-limiting examples of the present disclosure:

### EXAMPLES

### Example 1: Preparation of 3-(2-chlorophenyl)-5-methyl-N-(6-nitrobenzo[d]thiazol-2-yl)isoxazole-4-carboxamide (“B2”)

### I. Synthesis

### a) Synthesis of 2-chlorobenzaldehyde oxime

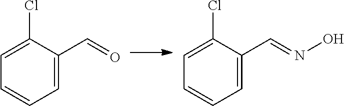 [SMILES: O=Cc1ccccc1Cl ; O/N=C/c1ccccc1Cl]

To a stirred mixture of 2-chlorobenzaldehyde (100 g, 0.711 mol) in water (100 mL) and 96% ethanol (250 mL) cooled to 5 °C by ice bath, hydroxylamine hydrochloride (54.5 g, 0.784 mol) was added. The solution was stirred at 5 °C and 50% NaOH (95 mL) was added slowly, turning the mixture strongly alkaline as indicated by litmus paper. The mixture was allowed to warm up to room temperature (r.t.), stirred for 1 h, acidified with concentrated HCl (25 mL) to strongly acidic as indicated by litmus paper, and extracted with CH₂Cl₂ (3×250 mL). The combined organic extracts were washed with water (2×250 mL), brine (2×250 mL), dried over anhydrous sodium sulfate, filtered, and concentrated to give a crystallizing oil. Yield: 109.0 g (98%). liquid chromatography-mass spectrometry (LC-MS): MS (electrospray ionization; ESI) calcd. for: C₇H₇ClNO [M+H]⁺ 156.01; found: 155.99, retention time (t_{R})=2.67 min.

### b) Synthesis of 2-chloro-N-hydroxybenzimidoyl chloride

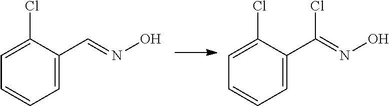 [SMILES: O/N=C/c1ccccc1Cl ; O/N=C(\Cl)c1ccccc1Cl]

To a stirred solution of the 2-chlorobenzaldehyde oxime (100 g, 0.64 mol) in N,N-dimethylformamide (DMF; 630 mL) was added one-fifth portion (17.1 g, 0.13 mol) of N-chlorosuccinimide (NCS). To induce the reaction, HCl(g) from an HCl generator was bubbled through the reaction mixture with vigorous stirring until the temperature started to increase. The temperature of the solution rose to 35 °C and was kept below this value by periodical ice bath cooling. The rest of the NCS (68.4 g, 0.51 mol) was added in 4 portions. The time interval between portions was 15 min and then the reaction mixture was stirred for 0.5 h. The solution was concentrated in vacuo, and the residue was dissolved in methyl t-butyl ether (MTBE; 600 mL), washed with water (2×500 mL), brine (1×500 mL), dried over anhydrous sodium sulfate (25 g), filtered, and concentrated in vacuo to give a yellowish oil. Yield: 116.8 g (96%). LC-MS: MS (ESI) calcd. for: C₇H₆Cl₂NO [M+H]⁺ 189.97; found: 190.00, t_{R}=3.14 min.

### c) Synthesis of 3-(2-chlorophenyl)-5-methylisoxazole-4-carboxylic acid

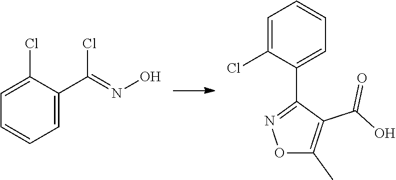 [SMILES: O/N=C(\Cl)c1ccccc1Cl ; Cc1onc(-c2ccccc2Cl)c1C(=O)O]

To a solution of 2-chloro-N-hydroxybenzimidoyl chloride (121.76 g, 0.64 mol) in methanol (900 mL), cooled between −15 to −20 °C, was added NaOH (1.3 g, 0.032 mol). Then sodium salt of methyl acetoacetate (88.44 g, 0.64 mol) was added in 10 portions with 10 min time interval between portions at −15 °C. The reaction mixture was subsequently allowed to heat up to 20 °C and stirred for an additional 30 min. Then the reaction mixture was cooled to 0 °C, a solution of NaOH (51.0 g, 1.28 mol) in water (150 mL) was added and the reaction mixture was stirred at 50 °C for 2 h. The methanol was removed in vacuo, the residual suspension was diluted with water (1500 mL) and the pH adjusted to 2.0-2.5 with 20% H₂SO₄ (250 mL). The compound was filtered, washed with water (2×500 mL) and dried in vacuo to give the product as yellow solids. Yield: 129.33 g (85%). LC-MS: MS (ESI) calcd. for: C₁₁H₉ClNO₃ [M+H]⁺ 238.02; found: 237.97, t_{R}=3.60 min. Nuclear magnetic resonance (NMR): ¹H NMR (DMSO-d₆, 500 MHz, δ, ppm): 2.72 (s, 3H), 7.42-7.48 (m, 2H), 7.50-7.54 (m, 1H), 7.57-7.59 (m, 1H), 12.98 (br. s, 1H). ¹³C NMR (DMSO-d₆, 125 MHz, δ, ppm): 12.85, 109.98, 127.01, 128.43, 129.27, 131.15, 131.26, 132.93, 160.62, 162.30, 175.06.

### d) Synthesis of 3-(2-chlorophenyl)-5-methyl-N-(6-nitrobenzo[d]thiazol-2-yl)isoxazole-4-carboxamide

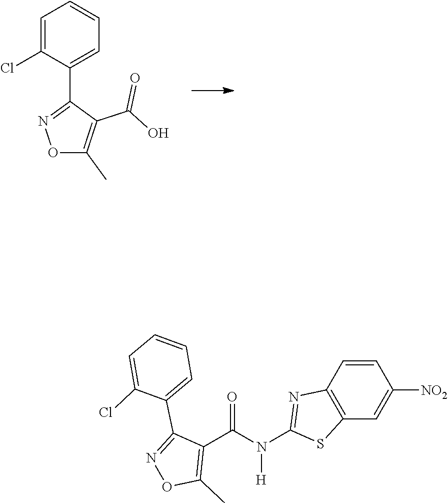 [SMILES: Cc1onc(-c2ccccc2Cl)c1C(=O)O ; Cc1onc(-c2ccccc2Cl)c1C(=O)Nc1nc2ccc([N+](=O)[O-])cc2s1]

To a solution of 3-(2-chlorophenyl)-5-methylisooxazole-4-carboxylic acid (100 g, 0.42 mol), 2-amino-6-nitrobenzothiazole (90.75 g, 0.46 mol, 1.1 eq) and ethyl cyanohydroxyiminoacetate (Oxyma; 68.25 g, 0.48 mol, 1.1 eq) in DMF (900 mL) were added triethylamine (70.0 mL, 0.50 mol, 1.2 eq) and diisopropylcarbodiimide (DIC) (85.0 mL, 0.55 mol, 1.3 eq). The reaction mixture was stirred at 50 °C under nitrogen atmosphere for 16 h, allowed to cool down to r.t., then kept in a fridge at 5 °C for 2 h. The resulting suspension was filtered, the filter cake was washed with DMF (2×50 ml), and the filtrate was concentrated in vacuo. The residue was dissolved in ethyl acetate (3000 mL), extracted with 2% K₂CO₃ in water (2×1000 mL), 0.2 M HCl (2×1000 mL), brine (2×1000 mL), dried over anhydrous sodium sulphate (50 g), filtered and concentrated. The product was dissolved in ethyl acetate (20 mL/g) under reflux and activated charcoal powder (1 g per 10 g of product) was added. The resulting suspension was heated to reflux for 10 minutes under intensive stirring, then filtered by hot filtration. The filtrate was heated to reflux, and heptane (10 mL/g) was added slowly. The solution was allowed to cool down to room temperature and stirred gently overnight. Yield: 121.7 g (70%). LC-MS: MS (ESI) calcd. for: C₁₈H₁₂C₁N₄O₄S [M+H]⁺ 415.02; found: 414.84, t_{R}=4.26 min. ¹H NMR (CDCl₃, 500 MHz, δ, ppm): 2.89 (s, 3H), 7.49-7.65 (m, 4H), 7.69 (d, J=9.0 Hz, 1H), 8.28 (dd, J=9.0, 2.3 Hz, 1H), 8.73 (d, J=2.3 Hz, 1H). ¹³C NMR (CDCl₃, 125 MHz, δ, ppm): 13.72, 110.21, 118.29, 120.92, 122.34, 126.36, 128.32, 131.02, 131.93, 132.42, 133.02, 133.96, 144.35, 152.02, 158.08, 159.46, 161.66, 177.08.

### II. Discussion

3-(2-chlorophenyl)-5-methyl-N-(6-nitrobenzo[d]thiazol-2-yl)isoxazole-4-carboxamide was obtained according to Scheme 2.

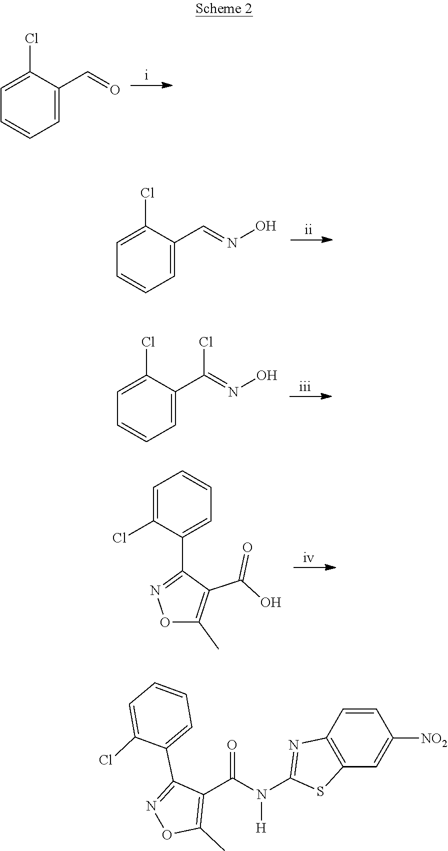

i) NH₂OH·HCl, NaOH, EtOH/water, 20 °C, 2 h; ii) NCS, DMF, 20 °C, 0.5 h; iii) sodium salt of methyl acetoacetate, NaOH, MeOH, −20 °C to +50 °C, 6 h; iv) 2-amino-6-nitrobenzothiazole, DIC, Oxyma, Et₃N, DMF, 50 °C, 16 h.

Briefly, 2-chlorobenzaldehyde oxime was prepared by treatment of 2-chlorobenzaldehyde with hydroxylamine hydrochloride in the presence of sodium hydroxide in ethanol. Then, by chlorination with N-chlorosuccinimide (NCS) in DMF, 2-chlorobenzaldehyde oxime was converted to 2-chloro-N-hydroxybenzimidoyl chloride. Cyclization of 2-chloro-N-hydroxybenzimidoyl chloride with sodium salt of methyl acetoacetate followed by hydrolysis with NaOH gave 3-(2-chlorophenyl)-5-methylisoxazole-4-carboxylic acid. Finally, amide coupling of 3-(2-chlorophenyl)-5-methylisoxazole-4-carboxylic acid with 2-amino-6-nitrobenzothiazole in the presence of diisopropylcarbodiimide (DIC) and Oxyma yielded 3-(2-chlorophenyl)-5-methyl-N-(6-nitrobenzo[d]thiazol-2-yl)isoxazole-4-carboxamide.

### Example 2: Biological Activity

### I. Materials and Methods

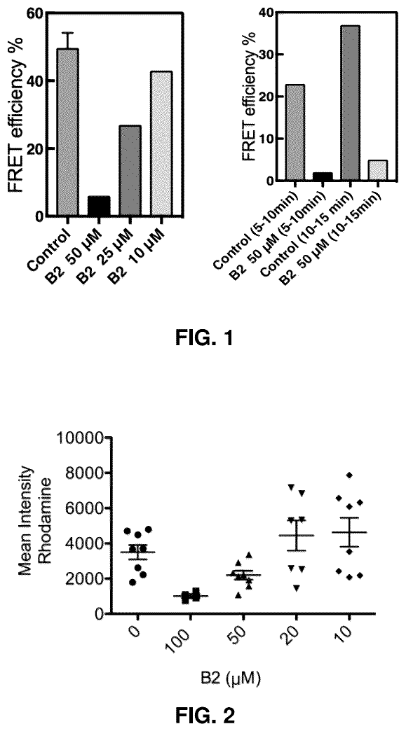

FACS-FRET of SWAP-70 in living cells: The method was in line with that previously described by Betaneli and Jessberger (2020). Briefly, two versions of SWAP-70, differently tagged with the fluorescent dyes Cerulean and Venus, were stably expressed in 293T cells. Upon activation of the cells, e.g., by sodium vanadate, SWAP-70 dimerizes. Thereby the two fluorophores associate and generate a fluorescence resonance energy transfer (FRET) signal, which is measured by FACS. FACS-FRET allows to analyze large numbers of cells simultaneously. The left plot in FIG. 1 shows inhibition of the FACS-FRET signal (FRET efficiency) and thus of SWAP-70 dimerization by, from left to right: 50, 25 and 10 μM B2 in comparison to control (far left), measured at 20 minutes after stimulation of the cells.

Neutrophil ROS production: Neutrophil reactive oxygen species (ROS) production was measured on human primary neutrophils, isolated from fresh blood samples using the MACSexpress™ neutrophil kit as described in the manufacturer's instructions (Miltenyi Biotec Inc.). Neutrophils in RPMI containing 50 μm dihydrorhodamine 123 (DHR-123) were triggered to produce ROS by treatment with 20 μm bacterial peptide N-formylmethionyl-leucyl-phenylalanine (fMLP) as a physiologically relevant stimulant. ROS were measured by FACS based on the conversion of DHR-123 to Rhodamine-123.

Neutrophil NETosis: Neutrophil production of extracellular traps, NETosis, was measured on human primary neutrophils, isolated from fresh blood samples as described above or by Pancoll gradient centrifugation. Neutrophils in RPMI at 4×10⁶ cells/ml were triggered to undergo NETosis in wells of a 96-well plate at a total cell number of 2×10⁵ cells per well. The cells were treated for 3 hours with either the ionophore phorbol myristate acetate (100 nM PMA) or the bacterial product pyocyanin (at 10 μM). Extracellular DNA was measured by fluorometry using the dye Sytox™ Green and/or by fluorescence microscopy.

B lymphocyte plasmablast development: For human B lymphocyte plasmablast development, primary human B cells were isolated from buffy coat using the MACSxpress Buffy Coat Pan B Cell Isolation Kit (Miltenyi Biotec) as per manufacturer's instructions. The cells were cultured in RPMI in the presence of human transferrin (20 μg/ml), IL-21 (100 ng/ml), recombinant hCD40L (100 μg/ml), anti-HA (50 μg/ml), at a cell density of 5×10⁴ cells/ml. Plasmablast formation after 5 days was measured by FACS using two sets of markers: CD38+/CD19low and CD138+ for two stages of plasmablast development.

B lymphocyte IgE production: B lymphocyte IgE production was measured in line with that previously described by Audzevich et al. (2013). Briefly, primary human B cells isolated by the MACSxpress Buffy Coat Pan B Cell Isolation Kit (Miltenyi Biotec) from buffy coat were stimulated for 6 days by recombinant hCD40L (100 μg/ml) and IL-4 (50 ng/ml) at a cell density of 5×10⁴ cells/ml. The frequency of IgE+B lymphocytes was determined by FACS at day 6 using anti CD19 and anti IgE antibodies.

Mast cell degranulation: Human primary mast cells were developed in StemSpan™ SFEM including 125 ng/ml SCF and 20 ng/ml IL3 from CD34+ hematopoietic precursor cells, and their purity after 4 to 5 weeks measured by FACS for c-kit and FceRI expression. This was measured by FACS using the appearance of Lamp-1 (CD107a) in line with that previously described by Klewer et al. (2021), applied here to human primary mast cells. Degranulation was triggered by preincubation of mast cells overnight with 1.3 μg/ml IgE followed by treatment with 0.2 μg/ml anti-IgE for 30 min.

Eosinophil ROS production: Human primary eosinophils were purified from buffy coat, firstly by density gradient centrifugation on ficoll to isolate total peripheral blood mononuclear cell (PBMC), and then by negative selection using the eosinophil purification kit from Miltenyi Inc. as per manufacturer's instructions. The eosinophils were then resuspended in pre-warmed tissue culture media, which had been left in the incubator overnight to equilibrate, containing 25 μM dihydrorhodamine 123 (DHR-123) at a cell density of 1×10⁶ cells/ml. The cells were then stimulated with phorbol 12-myristate 13-actetate (PMA) at a concentration of 1 μM in the presence or absence of B2 or DMSO for control in the incubator for 20 minutes and then immediately put on ice to stop the reaction. Levels of the oxidized DHR in the samples were measured by flow cytometry as described above in relation to neutrophil ROS production.

Mast cell cytokine release: Primary human mast cells developed in culture from CD34+ precursors as described above in relation to mast cell degranulation were stimulated at a cell density of 0.8×10⁶ cells/ml by 0.12 mg/ml IgE and 0.1 μg/ml anti-IgE in mast cell medium (StemSpan SFEM including 100 ng/ml SCF, 50 ng/ml IL6, 2 & FBS, and 1% Pen/Strep). Simultaneously, the cells were treated with 100 μM B2, and cytokine levels present in the culture supernatants were measured after 24 h. The levels of 105 cytokines were measured by the R&D Systems Human XL cytokine array as per manufacturer's instructions. The signals were measured by a fluorescence-based photometric read-out in the Li—COR Odyssey Fluorescent imaging system, and the spots quantified by Fiji software.

B lymphocyte cytokine release: Primary human B lymphocytes isolated from fresh buffy coat were stimulated at a cell density of 5×10⁵/ml in 1 ml by CD40L and IL4 for 5 days in a 24-well plate, and the levels of 105 cytokines in the cell culture supernatant were measured by the R&D Systems Human XL cytokine antibody array as per manufacturer's instructions. The signals were measured by a fluorescence-based photometric read-out in the Li—COR Odyssey Fluorescent imaging system, and the spots quantified by Fiji software.

Macrophage/monocyte migration: Monocytes/macrophages were purified from buffy coats, first by density gradient centrifugation on ficoll to isolate total PBMC, and then by negative selection using the monocyte purification kit from Miltenyi Inc. as per manufacturer's instructions. Monocytes were then resuspended in tissue culture media at a concentration of 5×10⁶ per ml. Monocyte migration to CCL2 was then measured using a Boyden chamber assay fitted with a membrane with 8 μM pores. This was performed as follows: 200 μl of cells were pipetted into the upper chamber in the presence or absence of B2 or DMSO control. In the lower chamber 1 ml of the chemokine solution at concentrations of 10 ng/mL and 100 ng/mL, together with the same amount of B2 or DMSO as in the upper chamber was added. Cells were then left to migrate for 6 hours and the number of cells migrating into the lower chamber was ascertained by standard cell counting.

Neutrophil adhesion to endothelial cells: Primary human pulmonary microvascular endothelial cells (HPMEC) were purchased from Promocell and maintained in endothelial cell media from Promocell as per the company's protocol. The endothelial cells were plated in flat bottom 96 well plastic plates at a concentration of 50,000 cells/well. After 24 h the endothelial cells were stimulated overnight by TNF-α (100 ng/ml) and the inhibitor. Neutrophils were isolated from whole blood using MACSxpress Neutrophil isolation kit. Neutrophils at a concentration of 1×10⁶ cells/ml were fluorescently labeled with 1 μM CellTracker™ dye CMFDA for 40 min at 37 °C. The labeled cells were resuspended in HBS solution (0.14 M NaCl, 5 mM KCl, 1 mM CaCl₂, 0.4 mM MgSO₄, 0.5 mM MgCl₂, 0.3 mM Na₂HPO₄, 6 mM Glucose, and 4 mM NaHCO₃). The stimulated endothelial cells were washed once with medium and 100 μl of labeled neutrophils and B2 or DMSO at indicated concentrations were added. Plates were incubated for 30 min at 37 °C, carefully washed two times with PBS to remove non-adherent cells. Adherent cells were fixed in 100% methanol for 10 min and visualized by inverted microscopy at 10× magnification. Samples were analyzed in triplicates and 3 fields of view were imaged per well. Cells per field of view were quantified using FiJi software.

Endothelial cell cytokine release: Primary human pulmonary microvascular endothelial cells (HPMEC) were purchased from Promocell and maintained in endothelial cell media from Promocell as per the company's protocol. To measure the release of inflammatory mediators HPMEC were cultured to confluence in a 12 well plate in 1 ml of endothelial cell media. Cells were then stimulated with TNFα at 100 ng/ml overnight in the presence or absence of B2 or control DMSO. The supernatant was then collected and inflammatory mediator levels were measured in it using the Cytokine XXL array from R&D systems according to manufacturer's instructions as described above.

### II. Results

B2 inhibits SWAP-70 dimerization in living cells (FIG. 1). Two versions of SWAP-70, differently tagged with the fluorescent dyes Cerulean and Venus, were stably expressed in 293T cells. Upon activation of the cells, e.g., by sodium vanadate, SWAP-70 dimerizes. Thereby the two fluorophores associate and generate a fluorescence resonance energy transfer (FRET) signal, which is measured by FACS. FACS-FRET allows to analyze large numbers of cells simultaneously. The left plot in FIG. 1 shows inhibition of the FACS-FRET signal (FRET efficiency) and thus of SWAP-70 dimerization by, from left to right: 50, 25 and 10 μM B2 in comparison to control (far left), measured at 20 minutes after stimulation of the cells. The right plot in FIG. 1 shows inhibition by 50 μM B2 at earlier time intervals (second column from left: 5-10 minutes; far right: 10-15 minutes) in comparison to controls (far left: 5-10 minutes; second column from right: 10-15 minutes) after stimulation. B2 was added to the cells 15 minutes prior to stimulation.

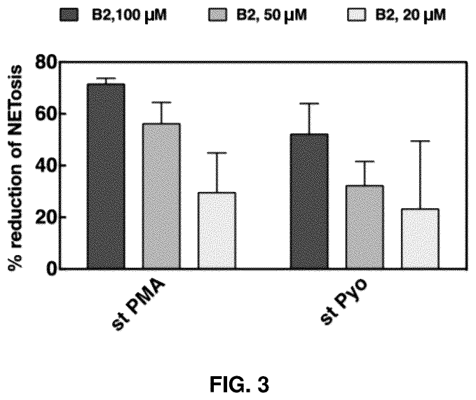

To investigate the effect of neutrophil ROS production by B2, human primary neutrophils, isolated from fresh blood samples, were triggered to produce reactive oxygen species (ROS) by treatment with the bacterial peptide N-formylmethionyl-leucyl-phenylalanine (fMLP) as a physiologically relevant stimulant and ROS were measured by FACS based on the conversion of dihydrorhodamine 123 to rhodamine 123. The effect of 4 different concentrations of B2 is shown (FIG. 2). There is clear inhibition of ROS production at 50 μM and 100 μM B2.

To investigate the effect of B2 on neutrophil NETosis, human primary neutrophils, isolated from fresh blood samples, were triggered to undergo NETosis (the expulsion of DNA in the environment) by treatment for 3 hours with either the ionophore phorbol myristate acetate (PMA) or the bacterial product pyocyanin. Extracellular DNA was measured by photometry using the dye Sytox™ Green. The results are shown in FIG. 3.

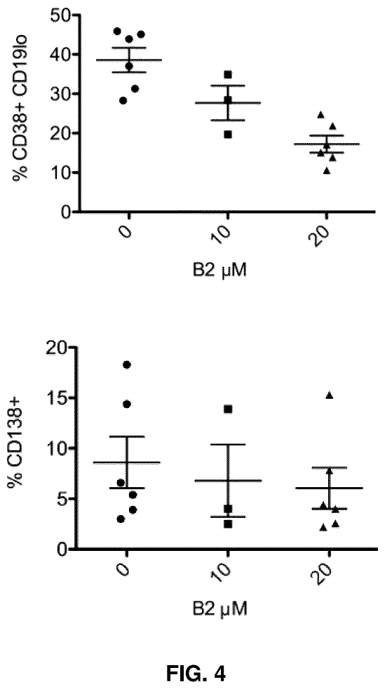

To investigate the inhibition of B lymphocyte plasmablast development by B2, primary human B cells, isolated from buffy coat, were cultured in the presence of CD40, IL21, anti-IgM, and plasmablast formation measured by FACS using two sets of markers: CD38+/CD19low (FIG. 4, upper plot) and CD138+(FIG. 4, lower plot) for two stages of plasmablast development.

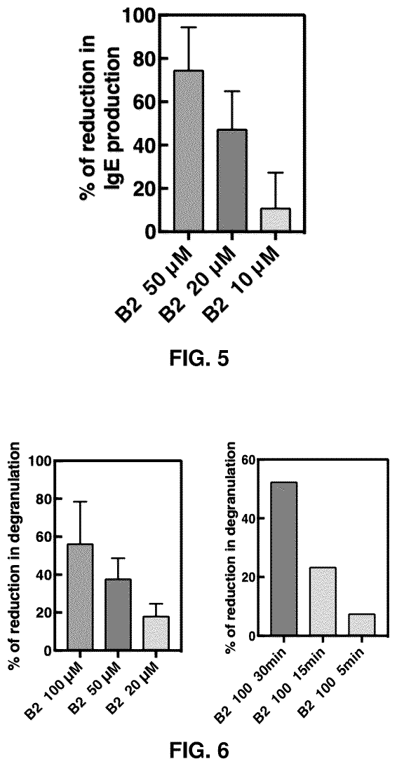

To investigate inhibition of B lymphocyte IgE production by B2, the frequency of IgE+B lymphocytes was determined by FACS at day 6 of primary human B cell cultures isolated from fresh buffy coat and stimulated by CD40L and IL4. The results are shown in FIG. 5. At 50 μM some toxicity was observed, not observed at lower concentrations.

To investigate the inhibition of mast cell degranulation by B2, degranulation was measured by FACS using the appearance of Lamp-1 (CD107a) on the surface of human primary mast cells upon degranulation. Degranulation was triggered by IgE/anti-IgE and B2 was applied 30 minutes before adding IgE. Three different concentrations of B2 were tested; from left to right in FIG. 6 (left plot): 100, 50 and 20 μM. The average of 11 experiments is shown as % reduction of degranulation. Degranulation was also measured at 3 time points after addition of 100 μM B2 (FIG. 6, right plot).

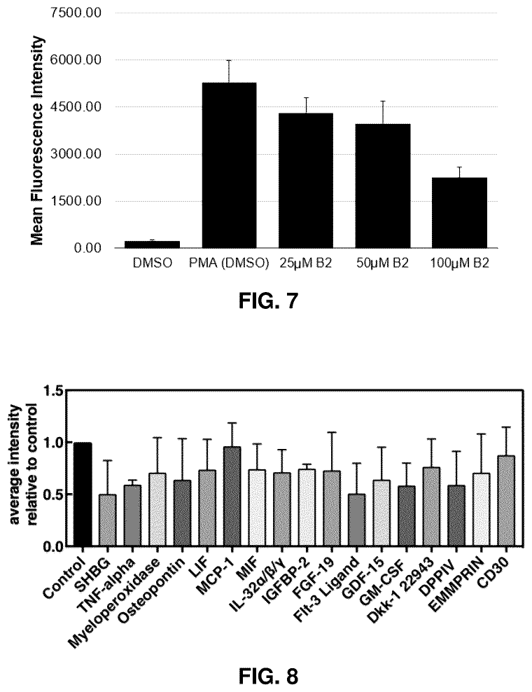

To investigate inhibition of eosinophil ROS production by B2, human primary eosinophils, isolated from fresh blood samples, were triggered to produce ROS by treatment with PMA, and ROS measured by FACS based on the conversion of dihydrorhodamine 123 to rhodamine 123 (FIG. 7).

To investigate the modulation of mast cell cytokine release by B2, primary human mast cells were stimulated by IgE/anti-IgE, treated with 100 μM B2, and cytokine levels present in the culture supernatants measured after 24 h. The levels of 105 cytokines were measured by the R&D Systems Human XL cytokine array and a fluorescence-based photometric read-out. Levels for exemplary cytokines relative to control are shown in FIG. 8.

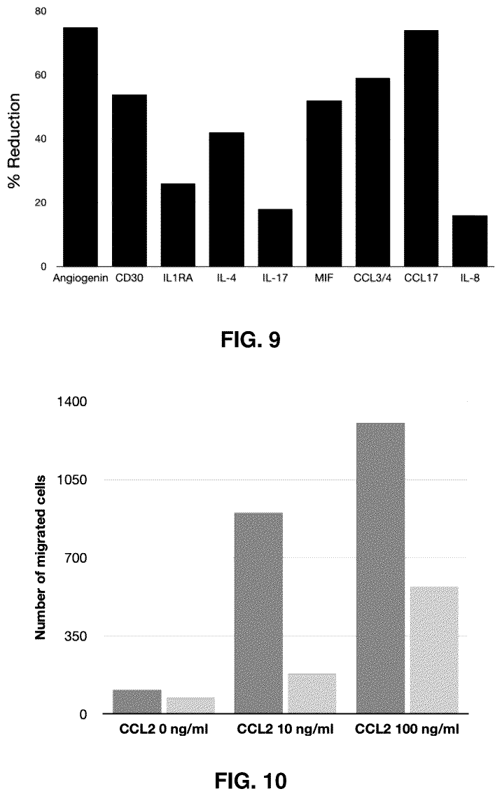

To investigate the modulation of B lymphocyte cytokine release by B2, primary human B lymphocytes isolated from fresh buffy coat were stimulated by CD40L and IL4 for 24 hours and the levels of 105 cytokines in the cell culture supernatant were measured by the R&D Systems Human XL cytokine array and a fluorescence-based photometric read-out. Levels for percent reduction in comparison to control are shown in FIG. 9.

To investigate the inhibition of macrophage/monocyte migration by B2, primary peripheral blood monocytes were isolated from buffy coat and subsequent negative selection. The cells were then tested in a Transwell™ set-up for migration towards the chemokine CCL2, known to play an important role in Covid-19 pathology. Migrated cells were quantified after 5 hours in the presence of carrier (DMSO) only or 50 μM B2 in DMSO (FIG. 10).

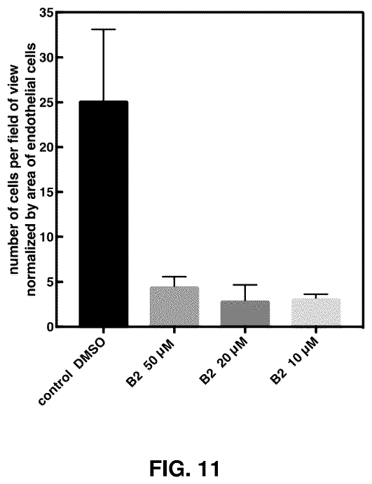

To investigate the adhesion of neutrophils to endothelial cells, human primary neutrophils, were fluorescently labeled with 1 μM CellTracker dye and resuspended in HBS solution prior to addition of B2 or DMSO. Green fluorescent adherent cells were counted, the area covered by endothelial cell was measured, and the number of neutrophils adherent to endothelial cells normalized for the area covered by endothelial cells. Data is displayed in FIG. 11 for control (DMSO) and three concentrations of B2 (10, 20, and 50 μM B2 in DMSO).

Table 1 shows a summary of the result of investigations of the modulation of stimulated pulmonary endothelial cell cytokine production by B2. Human primary pulmonary endothelial cells were triggered to produce cytokines by the pro-inflammatory cytokine TNFα. The levels of 105 cytokines in the cell culture supernatant were measured by the R&D Systems Human XL cytokine array and a fluorescence-based photometric read-out. The table lists cytokines whose production was reduced by >50% (left) or 20 to 50% upon treatment with 50 μM B2 (right).

TABLE 1

| Activated by TNF + >50% | Activated by TNF + 20-50% |
|---|---|
| Reduction at 50 μM | Reduction at 50 μM |
| VCAM-1 | Pentraxin-3 |
| CCL5/RANTES | CCL2/MCP-1 |
| CXCL1/GRO-alpha | CCL20/MIP-3 alpha |
| CXCL5/ENA-78 | CXCL10/IP10 |
| GMCSF | FGF19* |
| HGF | |
| ICAM-1 | |
| TFF3 | |

*Poor dose response, only minor increase with TNF.

Table 2 shows the inhibition of endothelial cell cytokine release by B2. Human primary coronary artery endothelial cells (HCAEC) were triggered to produce cytokines by the pro-inflammatory cytokine TNFα. The levels of 105 cytokines in the cell culture supernatant were measured by the R&D Systems Human XL cytokine array and a fluorescence-based photometric read-out. The table lists cytokines whose production was reduced by >50% (left) or 20 to 50% upon treatment with 50 μM B2 (right).

TABLE 2

| >50% inhibition by 50 μM B2 | 20-50% inhibition by 50 μM B2 |
|---|---|
| VCAM1 | CCL5 |
| Thrombospondin-1 | CXCL1 |
| CXCL10 | ICAM1 |
| CXCL11 | GM-CSF |
| IGFBP2 | |
| CXCL5 | |

### III. Discussion

The compound B2 was selected in vitro for inhibition of SWAP-70 and subsequently shown to block SWAP-70 dimerization, which is key to its activity (FIG. 1). B2 has been shown herein to inhibit the corresponding disease-relevant processes in distinct primary human leukocyte populations. B2 may be useful, for example, to reduce overshooting, pathological immune responses and inflammatory reactions while leaving the base immune system intact. Thus, B2 would not be considered an immune suppressor (such as corticosteroids, which come with serious side effects), but is a potent immune modulator. The inflammatory/immune processes targeted by B2 are thus not completely abolished but rather substantially reduced.

B2 inhibits IgE production by primary human peripheral blood B lymphocytes (FIG. 5). Thus, B2 and compounds of the disclosure may be useful as an inhibitor of IgE production. Human primary mast cells, developed from progenitors isolated from blood, are also inhibited by B2 in IgE-mediated degranulation (FIG. 6). Therefore, inhibition of SWAP-70 mediated by B2 and/or other compounds of the present disclosure may be useful to synergistically inhibit the two processes central to allergic reactions: IgE production and mast cell degranulation. This could be applied for treatment of various types of IgE-mediated allergies such as contact sensitivities, atopic dermatitis, food allergies, hay fever, venom allergies and others.

To our knowledge, no effective and safe immune modulator that prevents hyper-inflammation has been reported and applied for COVID-19 therapy and in related viral or non-viral inflammatory diseases. Since SWAP-70 is expressed in B cells, mast cells, neutrophils, eosinophils, and endothelial cells, its inhibition is expected to prevent severe COVID-19 disease. As described above the role of SWAP-70 in murine IgE production, mast cell degranulation and eosinophil activation has been reported. The data herein show, in addition, the inhibition of human primary B cell production of IgE (FIG. 5), of mast cell degranulation (FIG. 6), and of eosinophil activation (FIG. 7) by B2 treatment. Further, we have herein determined inhibition of human B cell plasmablast development by B2 (FIG. 4), which is consistent with a role of SWAP-70 in mouse plasmablast development (Chopin, Chacon-Martinez et al. 2011). In addition, neutrophil activities associated with COVID-19 and other viral lung inflammations are also inhibited by B2. Both ROS production and NETosis, which are COVID-19 disease drivers are substantially reduced (FIGS. 2 and 3, respectively). Furthermore, COVID-19 like other viral infections of the lung features heavy release of pro-inflammatory cytokines (a cytokine release syndrome, commonly known as the “cytokine storm”). We observed B2 to modulate the cytokine release profiles of mast cells, B cells and human pulmonary endothelial cells (FIGS. 8, 9, and Table 1, respectively), reducing specific pro-inflammatory cytokines. Macrophages or monocytes (from which macrophages develop) migrate into the infected lungs and trigger lung fibrosis during COVID-19 as described above. B2 reduces macrophage migration (FIG. 10) and thus will prevent build-up of fibrosis. Accordingly, B2 and other compounds of the disclosure may be useful for treatment of COVID-19 and other viral or non-viral inflammatory diseases. A six-fold synergistic effect beneficial for COVID-19 patients may be expected since B2 or similar compounds will reduce critical activities of B cells, plasmablasts, mast cells, neutrophils, eosinophils and monocytes/macrophages. Similar effects are expected for other viral infections of the upper airways and lungs such as RSV or influenza, which show overlapping characteristics. Preventing more severe lung damage is likely to reduce the risk of subsequent, opportunistic pneumonia, which may be caused by viruses like rhinovirus, influenza virus, or bacteria like Streptococcus pneumoniae.

Besides lung inflammation, other cells and tissues within an organism are negatively impacted by virus-triggered inflammation. The effects—often enhanced through the cytokine storm—can reach systemic dimensions, including many cell types and tissues such as but not limited to the gastrointestinal system, the kidneys, the heart, the nervous system, the skin and/or liver. In addition to viral infections, infections with other pathogens and other conditions can also cause severe lung and/or systemic pathologies where many of the cells and processes described above are involved. Examples are bacterial sepsis, infection with Yersinia pestis (plague), which triggers alveolar macrophages to produce excessive cytokines, or cytokine storm associated with autoimmune disorders (Fajgenbaum and June 2020). Given the inhibition of immune cell activation by B2 and the modulation of cytokine release, we expect treatment with B2 and/or other compounds of the present disclosure to be beneficial to a number of such diseases.

Similar to allergic reactions, many manifestations of asthma are also triggered by an IgE-allergen reaction, causing mast cell degranulation and subsequent inflammatory events, including eosinophil activation and migration. Following the logic described above in respect to allergies, the inhibition of IgE production and of mast cell degranulation by B2 and/or other compounds of the present disclosure is expected to substantially reduce asthma symptoms. Further, as described above, we observed reduced production of pro-inflammatory, potentially tissue-damaging reactive oxygen species (ROS) by human primary eosinophils isolated from blood when treated with B2 (FIG. 7). Therefore, we also expect B2 and/or other compounds of the present disclosure to reduce eosinophil migration and activation in the asthmatic lung setting. In addition, the finding of B2-mediated reduced secretion of certain cytokines, including chemokines, by human mast cells, B lymphocytes and pulmonary endothelial cells (FIGS. 8, 9, and Table 1, respectively) strongly suggest an additional benefit for an asthma patient with such treatment since the inflammatory milieu will be ameliorated, and decreased chemokine levels will reduce the influx of harmful immune cells into the asthmatic lung. Thus, a synergistic four-fold beneficial action of B2 or similar compounds for asthma patients may, be expected in acute asthma episodes and treatment with B2 and/or other compounds of the present disclosure may also be useful for prevention of such episodes.

Urticaria, a frequent skin disease, is typically caused by excessive mast cell degranulation. A central role of SWAP-70 in mast cell degranulation in mice as has been reported as described above and B2 has been demonstrated to inhibit degranulation of primary human mast cells (FIG. 6). In pseudo-allergic urticaria, IgE is involved and reducing IgE levels using anti IgE antibody therapy shows some success in some patients (Maurer, Altrichter et al. 2018, Wedi and Traidl 2021). This suggests that the effect of B2 on IgE production would be beneficial here as well. Corticosteroids are often the last resort for treatment in severe cases. B2 and/or other compounds of the present disclosure are expected to benefit such patients in acute episodes but also in chronic states, primarily through reducing mast cell degranulation but also by reducing eosinophil activation and thus generating a milieu of lowered sensitivity to stimuli. This establishment of chronic urticaria would be prevented by B2 and/or other compounds of the present disclosure. In an autoimmune variant of chronic urticaria, auto-antibody producing plasmablasts are centrally involved, whose generation would also be inhibited by B2 (FIG. 4) and would be predicted to be inhibited by other compounds of the present disclosure.

Without SWAP-70 osteoclasts cannot form a proper F-actin ring and therefore fail in bone resorption in cell culture and in mice. SWAP-70 deficient mice therefore become osteopetrotic, i.e., they show substantially increased bone mass. Thus, blocking SWAP-70 by treatment with B2 and/or other compounds of the present disclosure may reduce osteoclast activity and increase bone mass, thereby improving bone health in osteoporosis patients.

COPD involves hyper-activation of several of the immune cell types targeted by B2 as well as enhanced cytokine release and thus can also be classified as a special cytokine release syndrome (Brightling and Greening 2019). Other key immune cell types promoting COPD besides these two cell types are macrophages, mast cells and plasmablasts. As B2 reduces activity of all these cell types (FIGS. 2, 4, 6 and 7), we expect a significant synergistic benefit of B2 and/or other compounds of the present disclosure for treatment for COPD patients in both, acute episodes and chronic state, in the latter aiming at prevention of progressive disease.

Of the various forms of arthritis, inflammatory arthritis may also be a target of treatment with B2 and/or other compounds of the present disclosure. Rheumatoid arthritis (RA) is additionally characterized by production of autoantibodies by plasmablasts, which develop in this heavily pro-inflammatory environment, to which the plasmablast further contribute. B2 will inhibit plasmablast formation, neutrophil activation, macrophage migration and osteoclast formation and activity and will therefore benefit RA patients in a synergistic four-fold manner. This sets B2 apart from currently explored single cytokine blockers. Acute episodes but possibly also chronic, worsening RA will be addressed using B2 and/or other compounds of the present disclosure, which is expected to break the vicious cycle. Inflammatory arthritis in a broader sense also includes gout. In gout, urate crystals activate neutrophils to produce ROS, to perform NETosis and to secrete cytokines. B2 reduces at least ROS production and NETosis by human primary neutrophils as shown (FIGS. 2 and 3, respectively). RA is an autoimmune disease, and other autoimmune diseases may also be treatable with B2 and/or other compounds of the present disclosure. B2 and similar compounds would synergistically address at least three pathogenic mechanisms: IgE production, plasmablast development and NETosis. Similar considerations apply to other auto-antibody driven autoimmune diseases.

Cells centrally involved and hyper-activated during a cytokine storm include those addressed by B2: macrophages, mast cells, eosinophils, neutrophils, and endothelial cells. Their production of a range of pro-inflammatory cytokines including chemokines drives the storm. Reducing the production of such pro-inflammatory cytokines, chemokines most notably, by B2 and/or other compounds of the present disclosure is expected to significantly help in managing the acute cytokine release syndrome caused by a large variety of insults. B2 was shown herein to reduce cytokine secretion by endothelial cells, among the cytokines several chemokines are substantially reduced (Table 1). In addition, the secretion of several soluble adhesion molecules by endothelial cells is reduced by B2 (Table 1). Since such soluble adhesion molecules are associated with some forms of thrombosis (e.g., venous thromboembolism) (Torres, Matos et al. 2017), reducing the adhesion molecules will benefit in such and similar cases. Like in other inflammatory states, a vicious cycle of increased coagulation (thrombin) triggering increased endothelial cytokine production triggering more coagulation emerges that needs to be broken (Jackson, Darbousset et al. 2019). Table 2 shows the results for human primary coronary artery endothelial cells (HCAEC). B2 and other compounds of the present disclosure, by reducing endothelial cytokine production and immune cell recruitment (but not causing increased bleeding, a major side-effect of anti-coagulants) is expected to weaken or stop this cycle and thus inflammatory arterial thrombosis and other systemic inflammatory diseases that show a tendency for thrombosis.

SWAP-70 was confirmed as a causal gene for coronary artery disease in a more recent meta-study. Accordingly, inhibiting SWAP-70 by treatment with B2 and/or other compounds of the present disclosure would be predicted to benefit such patients, since leukocyte migration and accumulation and endothelial activation and cytokine release will be dampened by such treatment. In high-risk patients (40-60% heritability, wide range of other risk factors) treatment with B2 and/or other compounds of the present disclosure may also considered a preventive measure to reduce leukocyte accumulation.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR REFERENCES REFERRED TO IN THE DESCRIPTION

Altrichter, S., S. Frischbutter, J. S. Fok, P. Kolkhir, Q. Jiao, P. S. Skov, M. Metz, M. K. Church and M. Maurer (2020). “The role of eosinophils in chronic spontaneous urticaria.” J Allergy Clin Immunol 145(6): 1510-1516.
Audzevich, T., G. Pearce, M. Breucha, G. Gunal and R. Jessberger (2013). “Control of the STAT6-BCL6 antagonism by SWAP-70 determines IgE production.” J Immunol 190(10): 4946-4955.
Bahaie, N. S., M. R. Hosseinkhani, X. N. Ge, B. N. Kang, S. G. Ha, M. S. Blumenthal, R. Jessberger, S. P. Rao and P. Sriramarao (2012). “Regulation of eosinophil trafficking by SWAP-70 and its role in allergic airway inflammation.” J Immunol 188(3): 1479-1490.
Bastard, P., L. B. Rosen, Q. Zhang, E. Michailidis, H. H. Hoffmann, Y. Zhang, K. Dorgham, Q. Philippot, J. Rosain, V. Beziat, J. Manry, E. Shaw, L. Haljasmagi, P. Peterson, L. Lorenzo, L. Bizien, S. Trouillet-Assant, K. Dobbs, A. A. de Jesus, A. Belot, A. Kallaste, E. Catherinot, Y. Tandjaoui-Lambiotte, J. Le Pen, G. Kerner, B. Bigio, Y. Seeleuthner, R. Yang, A. Bolze, A. N. Spaan, O. M. Delmonte, M. S. Abers, A. Aiuti, G. Casari, V. Lampasona, L. Piemonti, F. Ciceri, K. Bilguvar, R. P. Lifton, M. Vasse, D. M. Smadja, M. Migaud, J. Hadjadj, B. Terrier, D. Duffy, L. Quintana-Murci, D. van de Beek, L. Roussel, D. C. Vinh, S. G. Tangye, F. Haerynck, D. Dalmau, J. Martinez-Picado, P. Brodin, M. C. Nussenzweig, S. Boisson-Dupuis, C. Rodriguez-Gallego, G. Vogt, T. H. Mogensen, A. J. Oler, J. Gu, P. D. Burbelo, J. I. Cohen, A. Biondi, L. R. Bettini, M. D'Angio, P. Bonfanti, P. Rossignol, J. Mayaux, F. Rieux-Laucat, E. S. Husebye, F. Fusco, M. V. Ursini, L. Imberti, A. Sottini, S. Paghera, E. Quiros-Roldan, C. Rossi, R. Castagnoli, D. Montagna, A. Licari, G. L. Marseglia, X. Duval, J. Ghosn, H. Lab, N.-U. I. R. t. C. Group, C. Clinicians, C.-S. Clinicians, C. G. Imagine, C. C. S. G. French, C. Milieu Interieur, V. C. C. Co, U. M. C. C.-B. Amsterdam, C. H. G. Effort, J. S. Tsang, R. Goldbach-Mansky, K. Kisand, M. S. Lionakis, A. Puel, S. Y. Zhang, S. M. Holland, G. Gorochov, E. Jouanguy, C. M. Rice, A. Cobat, L. D. Notarangelo, L. Abel, H. C. Su and J. L. Casanova (2020). “Autoantibodies against type I IFNs in patients with life-threatening COVID-19.” Science 370(6515).
Betaneli, V. and R. Jessberger (2020). “Mechanism of control of F-actin cortex architecture by SWAP-70.” J Cell Sci 133(2).
Borggrefe, T., S. Keshavarzi, B. Gross, M. Wabl and R. Jessberger (2001). “Impaired IgE response in SWAP-70-deficient mice.” Eur J Immunol 31(8): 2467-2475.
Borggrefe, T., M. Wabl, A. T. Akhmedov and R. Jessberger (1998). “A B-cell-specific DNA recombination complex.” J Biol Chem 273(27): 17025-17035.
Brightling, C. and N. Greening (2019). “Airway inflammation in COPD: progress to precision medicine.” Eur Respir J 54(2).
Chacon-Martinez, C. A., N. Kiessling, M. Winterhoff, J. Faix, T. Muller-Reichert and R. Jessberger (2013). “The switch-associated protein 70 (SWAP-70) bundles actin filaments and contributes to the regulation of F-actin dynamics.” J Biol Chem 288(40): 28687-28703.
Chopin, M., C. A. Chacon-Martinez and R. Jessberger (2011). “Fine tuning of IRF-4 expression by SWAP-70 controls the initiation of plasma cell development.” Eur J Immunol.
Chopin, M., L. Quemeneur, T. Ripich and R. Jessberger (2010). “SWAP-70 controls formation of the splenic marginal zone through regulating T1B-cell differentiation.” Eur J Immunol.
Church, M. K., P. Kolkhir, M. Metz and M. Maurer (2018). “The role and relevance of mast cells in urticaria.” Immunol Rev 282(1): 232-247.
Fajgenbaum, D. C. and C. H. June (2020). “Cytokine Storm.” N Engl J Med 383(23): 2255-2273.
Ferreira-Gomes, M., A. Kruglov, P. Durek, F. Heinrich, C. Tizian, G. A. Heinz, A. Pascual-Reguant, W. Du, R. Mothes, C. Fan, S. Frischbutter, K. Habenicht, L. Budzinski, J. Ninnemann, P. K. Jani, G. M. Guerra, K. Lehmann, M. Matz, L. Ostendorf, L. Heiberger, H. D. Chang, S. Bauherr, M. Maurer, G. Schonrich, M. Raftery, T. Kallinich, M. A. Mall, S. Angermair, S. Treskatsch, T. Dorner, V. M. Corman, A. Diefenbach, H. D. Volk, S. Elezkurtaj, T. H. Winkler, J. Dong, A. E. Hauser, H. Radbruch, M. Witkowski, F. Melchers, A. Radbruch and M. F. Mashreghi (2021). “SARS-CoV-2 in severe COVID-19 induces a TGF-beta-dominated chronic immune response that does not target itself.” Nat Commun 12(1): 1961.
Frangou, E., D. Vassilopoulos, J. Boletis and D. T. Boumpas (2019). “An emerging role of neutrophils and NETosis in chronic inflammation and fibrosis in systemic lupus erythematosus (SLE) and ANCA-associated vasculitides (AAV): Implications for the pathogenesis and treatment.” Autoimmun Rev 18(8): 751-760.
Garbe, A. I., A. Roscher, C. Schuler, A. H. Lutter, M. Glosmann, R. Bernhardt, M. Chopin, U. Hempel, L. C. Hofbauer, S. Rammelt, M. Egerbacher, R. G. Erben and R. Jessberger (2012). “Regulation of bone mass and osteoclast function depend on the F-actin modulator SWAP-70.” J Bone Miner Res.
Gross, B., T. Borggrefe, M. Wabl, R. R. Sivalenka, M. Bennett, A. B. Rossi and R. Jessberger (2002). “SWAP-70-deficient mast cells are impaired in development and IgE-mediated degranulation.” Eur J Immunol 32(4): 1121-1128.
Gustine, J. N. and D. Jones (2021). “Immunopathology of Hyperinflammation in COVID-19.” Am J Pathol 191(1): 4-17.
Jackson, S. P., R. Darbousset and S. M. Schoenwaelder (2019). “Thromboinflammation: challenges of therapeutically targeting coagulation and other host defense mechanisms.” Blood 133(9): 906-918.
Jang, S., E. J. Kwon and J. J. Lee (2022). “Rheumatoid Arthritis: Pathogenic Roles of Diverse Immune Cells.” Int J Mol Sci 23(2).
Kang, S. and T. Kishimoto (2021). “Interplay between interleukin-6 signaling and the vascular endothelium in cytokine storms.” Exp Mol Med 53(7): 1116-1123.
Kennedy, J. L., S. Pham and L. Borish (2019). “Rhinovirus and Asthma Exacerbations.” Immunol Allergy Clin North Am 39(3): 335-344.
Kilpatrick, L. E. and M. F. Kiani (2020). “Experimental Approaches to Evaluate Leukocyte-Endothelial Cell Interactions in Sepsis and Inflammation.” Shock 53(5): 585-595.
Klewer T, Bakic L, Miller-Reichert T, Kiewisz R, Jessberger G, Kiessling N, Roers A, Jessberger R. (2022). “E-Cadherin restricts mast cell degranulation in mice.” Eur J Immunol January; 52(1):44-53.
Kuri-Cervantes, L., M. B. Pampena, W. Meng, A. M. Rosenfeld, C. A. G. Ittner, A. R. Weisman, R. S. Agyekum, D. Mathew, A. E. Baxter, L. A. Vella, O. Kuthuru, S. A. Apostolidis, L. Bershaw, J. Dougherty, A. R. Greenplate, A. Pattekar, J. Kim, N. Han, S. Gouma, M. E. Weirick, C. P. Arevalo, M. J. Bolton, E. C. Goodwin, E. M. Anderson, S. E. Hensley, T. K. Jones, N. S. Mangalmurti, E. T. Luning Prak, E. J. Wherry, N. J. Meyer and M. R. Betts (2020). “Comprehensive mapping of immune perturbations associated with severe COVID-19.” Sci Immunol 5(49).
Kwon, Y. C., J. Lim, S. Y. Bang, E. Ha, M. Y. Hwang, K. Yoon, J. Y. Choe, D. H. Yoo, S. S. Lee, J. Lee, W. T. Chung, T. H. Kim, Y. K. Sung, S. C. Shim, C. B. Choi, J. B. Jun, Y. M. Kang, J. M. Shin, Y. K. Lee, S. K. Cho, B. J. Kim, H. S. Lee, K. Kim and S. C. Bae (2020). “Genome-wide association study in a Korean population identifies six novel susceptibility loci for rheumatoid arthritis.” Ann Rheum Dis 79(11): 1438-1445.
Liossis, S. N. and C. Staveri (2021). “What's New in the Treatment of Systemic Lupus Erythematosus.” Front Med (Lausanne) 8: 655100.
Lucas, C., P. Wong, J. Klein, T. B. R. Castro, J. Silva, M. Sundaram, M. K. Ellingson, T. Mao, J. E. Oh, B. Israelow, T. Takahashi, M. Tokuyama, P. Lu, A. Venkataraman, A. Park, S. Mohanty, H. Wang, A. L. Wyllie, C. B. F. Vogels, R. Earnest, S. Lapidus, I. M. Ott, A. J. Moore, M. C. Muenker, J. B. Fournier, M. Campbell, C. D. Odio, A. Casanovas-Massana, I. T. Yale, R. Herbst, A. C. Shaw, R. Medzhitov, W. L. Schulz, N. D. Grubaugh, C. Dela Cruz, S. Farhadian, A. I. Ko, S. B. Omer and A. Iwasaki (2020). “Longitudinal analyses reveal immunological misfiring in severe COVID-19.” Nature 584(7821): 463-469.
Martinez, F. D. and D. Vercelli (2013). “Asthma.” Lancet 382(9901): 1360-1372.
Mathew, D., J. R. Giles, A. E. Baxter, A. R. Greenplate, J. E. Wu, C. Alanio, D. A. Oldridge, L. Kuri-Cervantes, M. B. Pampena, K. D'Andrea, S. Manne, Z. Chen, Y. J. Huang, J. P. Reilly, A. R. Weisman, C. A. G. Ittner, O. Kuthuru, J. Dougherty, K. Nzingha, N. Han, J. Kim, A. Pattekar, E. C. Goodwin, E. M. Anderson, M. E. Weirick, S. Gouma, C. P. Arevalo, M. J. Bolton, F. Chen, S. F. Lacey, S. E. Hensley, S. Apostolidis, A. C. Huang, L. A. Vella, U. P. C. P. Unit, M. R. Betts, N. J. Meyer and E. J. Wherry (2020). “Deep immune profiling of COVID-19 patients reveals patient heterogeneity and distinct immunotypes with implications for therapeutic interventions.” bioRxiv.
Maurer, M., S. Altrichter, O. Schmetzer, J. Scheffel, M. K. Church and M. Metz (2018). “Immunoglobulin E-Mediated Autoimmunity.” Front Immunol 9: 689.
Merad, M., C. A. Blish, F. Sallusto and A. Iwasaki (2022). “The immunology and immunopathology of COVID-19.” Science 375(6585): 1122-1127.
Merad, M. and J. C. Martin (2020). “Pathological inflammation in patients with COVID-19: a key role for monocytes and macrophages.” Nat Rev Immunol 20(6): 355-362.
Nikpay, M., A. Goel, H. H. Won, L. M. Hall, C. Willenborg, S. Kanoni, D. Saleheen, T. Kyriakou, C. P. Nelson, J. C. Hopewell, T. R. Webb, L. Zeng, A. Dehghan, M. Alver, S. M. Armasu, K. Auro, A. Bjonnes, D. I. Chasman, S. Chen, I. Ford, N. Franceschini, C. Gieger, C. Grace, S. Gustafsson, J. Huang, S. J. Hwang, Y. K. Kim, M. E. Kleber, K. W. Lau, X. Lu, Y. Lu, L. P. Lyytikainen, E. Mihailov, A. C. Morrison, N. Pervjakova, L. Qu, L. M. Rose, E. Salfati, R. Saxena, M. Scholz, A. V. Smith, E. Tikkanen, A. Uitterlinden, X. Yang, W. Zhang, W. Zhao, M. de Andrade, P. S. de Vries, N. R. van Zuydam, S. S. Anand, L. Bertram, F. Beutner, G. Dedoussis, P. Frossard, D. Gauguier, A. H. Goodall, O. Gottesman, M. Haber, B. G. Han, J. Huang, S. Jalilzadeh, T. Kessler, I. R. Konig, L. Lannfelt, W. Lieb, L. Lind, C. M. Lindgren, M. L. Lokki, P. K. Magnusson, N. H. Mallick, N. Mehra, T. Meitinger, F. U. Memon, A. P. Morris, M. S. Nieminen, N. L. Pedersen, A. Peters, L. S. Rallidis, A. Rasheed, M. Samuel, S. H. Shah, J. Sinisalo, K. E. Stirrups, S. Trompet, L. Wang, K. S. Zaman, D. Ardissino, E. Boerwinkle, I. B. Borecki, E. P. Bottinger, J. E. Buring, J. C. Chambers, R. Collins, L. A. Cupples, J. Danesh, I. Demuth, R. Elosua, S. E. Epstein, T. Esko, M. F. Feitosa, O. H. Franco, M. G. Franzosi, C. B. Granger, D. Gu, V. Gudnason, A. S. Hall, A. Hamsten, T. B. Harris, S. L. Hazen, C. Hengstenberg, A. Hofman, E. Ingelsson, C. Iribarren, J. W. Jukema, P. J. Karhunen, B. J. Kim, J. S. Kooner, I. J. Kullo, T. Lehtimaki, R. J. F. Loos, O. Melander, A. Metspalu, W. Marz, C. N. Palmer, M. Perola, T. Quertermous, D. J. Rader, P. M. Ridker, S. Ripatti, R. Roberts, V. Salomaa, D. K. Sanghera, S. M. Schwartz, U. Seedorf, A. F. Stewart, D. J. Stott, J. Thiery, P. A. Zalloua, C. J. O'Donnell, M. P. Reilly, T. L. Assimes, J. R. Thompson, J. Erdmann, R. Clarke, H. Watkins, S. Kathiresan, R. McPherson, P. Deloukas, H. Schunkert, N. J. Samani and M. Farrall (2015). “A comprehensive 1,000 Genomes-based genome-wide association meta-analysis of coronary artery disease.” Nat Genet 47(10): 1121-1130.
Ocana-Morgner, C., P. Reichardt, M. Chopin, S. Braungart, C. Wahren, M. Gunzer and R. Jessberger (2011). “Sphingosine 1-Phosphate-Induced Motility and Endocytosis of Dendritic Cells Is Regulated by SWAP-70 through RhoA.” J Immunol 186(9): 5345-5355.
Pearce, G., V. Angeli, G. J. Randolph, T. Junt, U. von Andrian, H. J. Schnittler and R. Jessberger (2006). “Signaling protein SWAP-70 is required for efficient B cell homing to lymphoid organs.” Nat Immunol 7(8): 827-834.
Pearce, G., T. Audzevich and R. Jessberger (2011). “SYK regulates B-cell migration by phosphorylation of the F-actin interacting protein SWAP-70.” Blood 117(5): 1574-1584.
Roscher, A., T. Hasegawa, S. Dohnke, C. Ocana-Morgner, N. Amizuka, R. Jessberger and A. I. Garbe (2016). “The F-actin modulator SWAP-70 controls podosome patterning in osteoclasts.” Bone Rep 5: 214-221.
Saini, S. S. and A. P. Kaplan (2018). “Chronic Spontaneous Urticaria: The Devil's Itch.” J Allergy Clin Immunol Pract 6(4): 1097-1106.
Sivalenka, R. R. and R. Jessberger (2004). “SWAP-70 regulates c-kit-induced mast cell activation, cell-cell adhesion, and migration.” Mol Cell Biol 24(23): 10277-10288.
Sivalenka, R. R., M. Sinha and R. Jessberger (2008). “SWAP-70 regulates mast cell FecepsilonRI-mediated signaling and anaphylaxis.” Eur J Immunol 38(3): 841-854.
Torres, C., R. Matos, S. Morais, M. Campos and M. Lima (2017). “Soluble endothelial cell molecules and circulating endothelial cells in patients with venous thromboembolism.” Blood Coagul Fibrinolysis 28(8): 589-595.
Touma, Z. and D. D. Gladman (2017). “Current and future therapies for SLE: obstacles and recommendations for the development of novel treatments.” Lupus Sci Med 4(1): e000239.
Veras, F. P., M. C. Pontelli, C. M. Silva, J. E. Toller-Kawahisa, M. de Lima, D. C. Nascimento, A. H. Schneider, D. Caetite, L. A. Tavares, I. M. Paiva, R. Rosales, D. Colon, R. Martins, I. A. Castro, G. M. Almeida, M. I. F. Lopes, M. N. Benatti, L. P. Bonjorno, M. C. Giannini, R. Luppino-Assad, S. L. Almeida, F. Vilar, R. Santana, V. R. Bollela, M. Auxiliadora-Martins, M. Borges, C. H. Miranda, A. Pazin-Filho, L. L. P. da Silva, L. D. Cunha, D. S. Zamboni, F. Dal-Pizzol, L. O. Leiria, L. Siyuan, S. Batah, A. Fabro, T. Mauad, M. Dolhnikoff, A. Duarte-Neto, P. Saldiva, T. M. Cunha, J. C. Alves-Filho, E. Arruda, P. Louzada-Junior, R. D. Oliveira and F. Q. Cunha (2020). “SARS-CoV-2-triggered neutrophil extracellular traps mediate COVID-19 pathology.” J Exp Med 217(12).
Wendisch, D., O. Dietrich, T. Mari, S. von Stillfried, I. L. Ibarra, M. Mittermaier, C. Mache, R. L. Chua, R. Knoll, S. Timm, S. Brumhard, T. Krammer, H. Zauber, A. L. Hiller, A. Pascual-Reguant, R. Mothes, R. D. Bulow, J. Schulze, A. M. Leipold, S. Djudjaj, F. Erhard, R. Geffers, F. Pott, J. Kazmierski, J. Radke, P. Pergantis, K. Bassler, C. Conrad, A. C. Aschenbrenner, B. Sawitzki, M. Landthaler, E. Wyler, D. Horst, C.-O. I. Deutsche, S. Hippenstiel, A. Hocke, F. L. Heppner, A. Uhrig, C. Garcia, F. Machleidt, S. Herold, S. Elezkurtaj, C. Thibeault, M. Witzenrath, C. Cochain, N. Suttorp, C. Drosten, C. Goffinet, F. Kurth, J. L. Schultze, H. Radbruch, M. Ochs, R. Eils, H. Muller-Redetzky, A. E. Hauser, M. D. Luecken, F. J. Theis, C. Conrad, T. Wolff, P. Boor, M. Selbach, A. E. Saliba and L. E. Sander (2021). “SARS-CoV-2 infection triggers profibrotic macrophage responses and lung fibrosis.” Cell 184(26): 6243-6261 e6227.
Wu, M. L., F. L. Liu, J. Sun, X. Li, X. Y. He, H. Y. Zheng, Y. H. Zhou, Q. Yan, L. Chen, G. Y. Yu, J. Chang, X. Jin, J. Zhao, X. W. Chen, Y. T. Zheng and J. H. Wang (2021). “SARS-CoV-2-triggered mast cell rapid degranulation induces alveolar epithelial inflammation and lung injury.” Signal Transduct Target Ther 6(1): 428.
Zaki, S. R. and C. D. Paddock (2008). “Viral infections of the lung.” Dail and Hammar's Pulmonary Pathology. 426-75.
Zheng, Q., Y. Ma, S. Chen, Q. Che and D. Chen (2020). “The Integrated Landscape of Biological Candidate Causal Genes in Coronary Artery Disease.” Front Genet 11: 320.

What is claimed is:

1-44. (canceled)

45. A method of:
treating an inflammatory disease, disorder or condition;
treating a disease, disorder or condition treatable by inhibiting SWAP-70; or
of treating a disease, disorder or condition treatable by inhibiting neutrophil reactive oxygen species (ROS) production, neutrophil NETosis, B lymphocyte plasmablast development, B lymphocyte IgE production, mast cell degranulation, eosinophil ROS production, mast cell cytokine release, B lymphocyte cytokine release, endothelial cell cytokine release, endothelial cell-immune cell interactions, and/or macrophage/monocyte migration,
the method comprising administering an effective amount of a compound of Formula I or a pharmaceutically acceptable salt, solvate and/or prodrug thereof to a subject in need thereof: 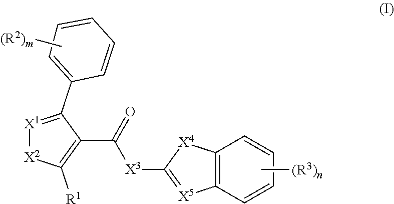
wherein
R¹ is selected from H, C₁₋₁₀alkyl, substituted or unsubstituted aryl and heteroaryl;
R² and R³ are each independently selected from C₁₋₁₀alkyl, substituted or unsubstituted aryl, heteroaryl, halo, OR⁴, NO₂ and SO₂R⁵;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is an integer of from 0 to 5; and
n is an integer of from 0 to 4.

46. The method of claim 45, wherein m is 1 and/or wherein n is 1.

47. The method of claim 45, wherein:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

48. The method of claim 45, wherein R¹ is C₁₋₄alkyl and/or wherein R² is halo and/or wherein R³ is NO₂.

49. The method of claim 45, wherein R¹ is methyl and/or wherein R² is chloro.

50. The method of claim 45, wherein X¹ is N and/or wherein X² is O and/or wherein X³ is NH and/or wherein X⁴ is S and/or wherein X⁵ is N.

51. The method of claim 45, wherein the compound of Formula I has the structure:

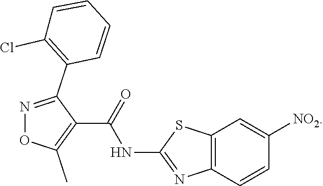 [SMILES: Cc1onc(-c2ccccc2Cl)c1C(=O)Nc1nc2ccc([N+](=O)[O-])cc2s1]

52. The method of claim 45, wherein the subject is a human.

53. The method of claim 45, wherein the disease, disorder or condition is a microbial infection, asthma, systemic inflammation, endothelial inflammation, an allergy, urticaria, osteoporosis, chronic obstructive pulmonary disease (COPD), arthritis, an autoimmune disorder, a cytokine release syndrome, avascular necrosis and/or sepsis.

54. The method of claim 53, wherein the microbial infection is a viral infection, and the treatment is for virus-induced lung inflammation.

55. The method of claim 53, wherein the compound of Formula I or the pharmaceutically acceptable salt thereof is for treatment of disease caused by the SARS-CoV-2 virus.

56. The method of claim 45, wherein the compound of Formula I is administered via inhalation.

57. A pharmaceutical composition comprising a compound of Formula I(b) or a pharmaceutically acceptable salt, solvate and/or prodrug thereof: 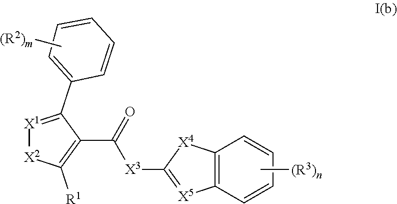
wherein
R¹ is selected from H, C₁₋₁₀alkyl, substituted or unsubstituted aryl and heteroaryl;
R² and R³ are each independently selected from C₁₋₁₀alkyl, substituted or unsubstituted aryl, heteroaryl, halo, OR⁴, NO₂ and SO₂R⁵;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is an integer of from 0 to 5; and
n is an integer of from 0 to 4;
provided that:
when m is 0, R³ is selected from substituted or unsubstituted aryl, heteroaryl, halo, NO₂ and SO₂R⁵;
when m is 1 and R² is halo, n is an integer of from 1 to 4, and R³ is selected from substituted or unsubstituted aryl, heteroaryl and NO₂; and
when m is 2, R² is selected from C₁₋₁₀alkyl, substituted or unsubstituted aryl, heteroaryl, OR⁴, NO₂ and SO₂R⁵.

58. The pharmaceutical composition of claim 57, wherein m is 1 and/or wherein n is 1.

59. The pharmaceutical composition of claim 57, wherein:
R¹ is selected from H, C₁₋₁₀alkyl, substituted or unsubstituted aryl and heteroaryl;
R² is selected from C₁₋₁₀alkyl, substituted or unsubstituted aryl, heteroaryl, halo, OR⁴, NO₂ and SO₂R⁵;
R³ is NO₂;
X¹ is selected from N and CH;
X² is selected from O, S and NR⁶;
X³ is selected from O and NR⁷;
X⁴ is selected from O, S, CH₂, SO, SO₂ and NR⁸;
X⁵ is selected from N and CH;
R⁴, R⁵, R⁶, R⁷ and R⁸ are each independently selected from H, C₁₋₁₀alkyl, substituted or unsubstituted aryl and heteroaryl;
m is 1; and
n is an integer of from 1 to 4.

60. The pharmaceutical composition of claim 57, wherein R¹ is C₁₋₄alkyl and/or wherein R² is halo and/or wherein R³ is NO₂.

61. The pharmaceutical composition of claim 57, wherein R¹ is methyl and/or wherein R² is chloro.

62. The pharmaceutical composition of claim 57, wherein X¹ is N and/or wherein X² is O and/or wherein X³ is NH and/or wherein X⁴ is S and/or wherein X⁵ is N.

63. The pharmaceutical composition of claim 57, wherein: R¹ is C₁₋₅alkyl, R² is halo, R³ is NO₂, X¹ is N, X² is O, X³ is NH, X⁴ is S, X⁵ is N, m is 1 and n is 1.

64. The pharmaceutical composition of claim 57, wherein the compound of Formula I(b) has the structure:

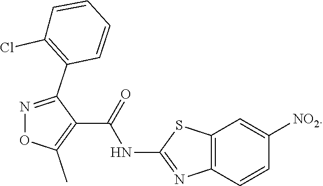 [SMILES: Cc1onc(-c2ccccc2Cl)c1C(=O)Nc1nc2ccc([N+](=O)[O-])cc2s1]

65. The pharmaceutical composition of claim 57, further comprising a pharmaceutically acceptable carrier.
